(19) United States
(12) Patent Application Publication
(10) Pub. No.: US 2026/0284339 A1
(43) Pub. Date: Sep. 24, 2026

(54) BLOWER FOR PORTABLE/WEARABLE RPT DEVICE

(71) Applicant: RESMED MOTOR TECHNOLOGIES INC., Chatsworth, CA (US)

(72) Inventors: Barton John KENYON, Sydney (AU); Jeegarkumar Subhashchandra KAPADIA, Sydney (AU); Christopher Scott EDWARDS, Canoga Park, CA (US); Sung Hwa JEUNG, Chatsworth, CA (US); Emily Claire SHRUBB, Sydney (AU)

(21) Appl. No.: 19/480,154

(22) PCT Filed: Apr. 19, 2024
(86) PCT No.: PCT/US2024/025413

§ 371 Date: Oct. 30, 2025

Related U.S. Application Data
(60) Provisional application No. 63/463,128, filed on May 1, 2023.
(60) Provisional application No. 63/632,092, filed on Apr. 10, 2024.

(51) Int. Cl. A61M 16/00

(52) U.S. Cl. CPC A61M 16/0066; A61M 2205/0216; A61M 2205/103; A61M 2205/3331; A61M 2205/3606; A61M 2205/42; A61M 2206/20

(57) ABSTRACT

A blower comprises a motor configured to drive a shaft, a first housing including a first inlet, a first impeller configured to be rotated by the shaft to deliver a first flow of air from the first inlet toward an outlet, the first impeller including impeller blades each of which is curved along at least a portion of its length such that each blade curves away from a direction of rotation of the first impeller, a second housing including a second inlet, and a second impeller configured to be rotated by shaft to deliver a second flow of air from the second inlet toward the outlet, the second impeller including impeller blades each of which is curved along at least a portion of its length such that each of blade curves away from a direction of rotation of the second impeller, wherein the first flow and the second flow are configured to flow parallel and opposite to one another along at least a portion of their flow path toward the outlet.

### 1 CROSS-REFERENCE TO RELATED APPLICATIONS

This application claims priority to U.S. Provisional Application Nos. 63/463,128, filed May 1, 2023, and 63/632,092, filed Apr. 10, 2024, each of which is incorporated herein by reference in its entirety.

Also, PCT Application No. PCT/AU2022/051321, filed Nov. 4, 2022, is incorporated herein by reference in its entirety.

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

Various respiratory illnesses, like sleep apnea, may be treated by providing positive pressurized air to the respiratory system of the body. When treating these illnesses, a sufficient flow rate must be applied in order to achieve a therapeutically effective pressure that assists in treating the illness and allows the patient to breathe more normally.

As part of treating the illness, the treatment system is ideally designed in order to promote patient comfort. For example, the more comfortable that the treatment system or device is to use, the more likely a patient will continue to use the treatment and improve or manage their illness. One way to promote patient comfort is to design smaller and/or lighter devices. This may reduce the feeling of constraint in a patient (e.g., by being tethered to cords or wires, and/or by wearing heavy items).

For example, improvements may be sought over the device illustrated in FIG. 2, which shows that the patient's range of motion is constrained as a result of a fixed RPT device and the various connecting wires and tubes. Reducing the size of the RPT device and positioning the RPT device on the positioning and stabilising structure worn by the patient may help to improve the patient's range of motion, and therefore the patient's compliance with the therapy.

When an RPT device is positioned on a positioning and stabilising structure to be worn on the patient's head, the device's size must be reduced in order to fit in the smaller area and to reduce the weight that the patient must support. As a result of this decrease in size of the overall RPT device, the impeller size may also decrease. A smaller impeller may generate less airflow, and therefore less pressure, as compared to a larger impeller operated at a similar speed. Accordingly, the RPT device must be designed so that smaller impeller(s) can generate a sufficient pressure so as to be therapeutically effective. Additional challenges related to wearable RPT devices include: reaching size point where other changes simply can't counteract the decrease in surface area (surface area inflection point); and modifications to increase surface area without overall size must be counterbalanced against enhanced noise/vibration concerns when wearable vs. tableside RPT devices.

The pressure generated by an RPT device may be increased in a variety of ways. For example, the rotational speed of the impeller may increase. Alternatively or in addition, multiple impellers may be used. Further alternatively or additionally, the size of the impellers may be increased. These changes should be balanced against increasing weight and/or noise that could otherwise limit patient compliance.

Some examples use multiple impellers arranged in series in order to generate greater airflow that is sufficient to be a therapeutically effective airflow. However, adding multiple impellers may require a longer motor shaft, which in turn may require a larger blower housing. As mentioned above, it may be more desirable to use a compact blower. Thus, adding too many additional impellers in order to achieve a desired pressure may form a blower that is too large to be supported by the patient's head.

Additionally, using a longer motor shaft in order to accommodate additional impellers may form a more flexible shaft. At high rotational speeds necessary for outputting therapeutically effective pressure causes vibrations along the shaft, which may impact performance.

Designing shorter and stiffer motor shafts may therefore reduce or eliminate some of the vibrational issues caused by using a longer and more flexible motor shaft at high operating speeds.

Therefore, alternative designs may use fewer but slightly larger impellers in order to achieve the therapeutically effective pressure.

Additionally, the arrangement of the individual impellers may be altered in order to increase the flow rate. For example, at least one impeller may be arranged on either side of a blower outlet so that the impellers produce parallel flows of air which may assist in delivering sufficient airflow while using smaller impellers than a series-only arrangement.

However, operation of the blower in the parallel arrangement may produce a fluttering affect because of the parallel flow paths, i.e., an imbalance in flow between each side of the blower. For example, some impellers may have a substantially flat fan curve (e.g., pressure vs. flow) which allows the impeller to achieve a desired outlet pressure at different flows. The fluttering effect can occur when such impellers in the blower in the parallel arrangement, although driven at the same speed, produce different flows. Ideally, each side of the blower in the parallel arrangement produces exactly half the desired flow rate. However, in practice, the substantially flat fan curve means each side is capable of producing many possible flow rates, and oscillating between those conditions, causing instability and noise.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a respiratory pressure therapy (RPT) system that includes a patient interface and a pressure generator, wherein the pressure generator is arranged to provide pressurised air to a patient wearing the patient interface.

The patient interface may include a seal-forming structure for sealing around a patient's airways and a plenum chamber for receiving the flow of pressurised air.

Another aspect of one form of the present technology is the impeller for a pressure generator, wherein the impeller rotates in order to generate a flow of pressurised air.

In one form, the impeller includes a conical or frustoconical shape and/or at least one blade of the impeller extends along a curvilinear path.

Another aspect of one form of the present technology is a pair of impellers connected in parallel to a motor.

Another aspect of one form of the present technology is a respiratory pressure therapy (RPT) system that includes a patient interface and a pressure generator, wherein the pressure generator is supported on the patient's head in use by the patient interface.

Another aspect of one form of the present technology is an impeller for a blower of a respiratory therapy system, the impeller comprising: a top shroud including a hub configured to be connected to a shaft of the motor of the blower; impeller blades; and a bottom shroud.

In some forms, the impeller is a mixed flow (or conical) impeller.

Another aspect of the present technology relates to a blower including a motor configured to drive a motor shaft, the motor shaft having a first end and a second end extending opposite the first end, a first housing including a first inlet, a first impeller connected to the first end of the motor shaft, the first impeller configured to be rotated by the motor shaft to deliver a first flow of air from the first inlet toward an outlet, the first impeller including a plurality of impeller blades, each of the impeller blades is curved along at least a portion of its length such that each of the impeller blades curves away from a direction of rotation of the first impeller and a top shroud and a bottom shroud that define a flow passage therebetween through the first impeller, the top shroud including a center opening which provides an impeller inlet to allow air to enter the flow passage of the first impeller, a second housing including a second inlet, and a second impeller connected to the second end of the motor shaft, the second impeller configured to be rotated by the motor shaft to deliver a second flow of air from the second inlet toward the outlet, the second impeller including a plurality of impeller blades, each of the impeller blades is curved along at least a portion of its length such that each of the impeller blades curves away from a direction of rotation of the second impeller and a top shroud and a bottom shroud that define a flow passage therebetween through the second impeller, the top shroud including a center opening which provides an impeller inlet to allow air to enter the flow passage of the second impeller, wherein the first flow generated by the first impeller and the second flow generated by the second impeller are configured to flow parallel and opposite to one another along at least a portion of their flow path toward the outlet, and the first and second flows are combined at the outlet, and wherein the flow passage of each of the first impeller and the second impeller is configured to produce a mixed flow of air having both an axial and centrifugal component.

In some forms, at least some of the plurality of blades of the first impeller and at least some of the plurality of blades of the second impeller include a concave surface and a convex surface, and wherein the convex surface is a leading surface oriented towards the direction of rotation.

In some forms, each blade of the plurality of blades includes a constant curvature.

In some forms, the plurality of blades for each of the first and second impellers includes primary blades and secondary blades, and wherein each of the primary blades are longer than the secondary blades.

In some forms, each of the secondary blades are disposed between a pair of primary blades, and wherein each of the primary blades are disposed between a pair of secondary blades.

In some forms, each of the first impeller and the second impeller includes a hub, and wherein the primary blades extend substantially between the hub and an outer perimeter of each impeller.

In some forms, the secondary blades extend substantially to the outer perimeter of each impeller and are spaced apart from the hub.

In some forms, the primary blades and the secondary blades have substantially the same radius of curvature.

In some forms, the first impeller and the second impeller each include an outer diameter between about 20 mm to about 30 mm.

In some forms, the first impeller and the second impeller are conical or frustoconical in shape.

In some forms, the first impeller and the second impeller are substantially identical.

In some forms, the first inlet and the second inlet are coaxial, and the outlet is generally perpendicular to the first and second inlets.

In some forms, the top shroud and the bottom shroud comprise two separate parts.

In some forms, the plurality of impeller blades comprise a once piece construction with the top shroud.

In some forms, the blower further comprises a first stator arranged downstream of the first impeller and a second stator arranged downstream of the second impeller.

In some forms, each of the first and second stators comprises stator vanes to direct the flow of air from the first and second impellers to the outlet.

In some forms, each of the stator vanes includes a surface configured to redirect the flow of air from a generally axial direction to a generally radial direction at the outlet.

In some forms, the motor includes a magnet mounted on the motor shaft and a stator assembly that surrounds the motor shaft and the magnet thereof, wherein the first and second stators are configured to support and maintain the stator assembly in an operative position, and wherein each of the first and second stators comprises a plurality of openings configured and arranged to expose at least a portion of the stator assembly to the flow of air to allow cooling of the stator assembly.

In some forms, the blower further comprises a pair of bearings to rotatably support the motor shaft, wherein one of the first and second stators comprises a pair of bearing seats configured to support and retain a respective one of the pair of bearings, and wherein each of the pair of bearing seats comprises an elastomeric material.

In some forms, each of the pair of bearing seats comprises one or more bumps or ribs configured to engage an outer race of the respective one of the pair of bearings.

In some forms, the blower further comprises a pair of bearings to rotatably support the motor shaft, wherein the motor includes a magnet mounted on the motor shaft and a stator assembly that surrounds the motor shaft and the magnet thereof, wherein one of the first and second stators comprises a bearing tube, and wherein the stator assembly is provided along an exterior surface of the bearing tube and the pair of bearings are provided along an interior of the bearing tube to support the motor shaft and the magnet within the interior.

In some forms, the blower further comprises a pair of bearings to rotatably support the motor shaft, wherein each of the pair of bearings is in the form of a rolling element bearing comprising ceramic ball bearings.

In some forms, the blower further comprises a pair of bearings to rotatably support the motor shaft, wherein the motor includes a magnet mounted on the motor shaft and a stator assembly that surrounds the motor shaft and the magnet thereof, and the blower further comprises a pair of spacers, each of the pair of spacers arranged between the magnet and a respective one of the pair of bearings, and wherein the motor shaft, the magnet, the pair of bearings, and the pair of spacers form a sub-assembly.

In some forms, the sub-assembly comprises a rotor-level balanced construction prior to connection of the first impeller and the second impeller to respective first and second ends of the motor shaft.

In some forms, the rotor-level balanced construction comprises mass removal from at least one of two planes each of which extends through a respective one of the pair of spacers.

In some forms, the sub-assembly, the first impeller connected to the first end of the motor shaft, and the second impeller connected to the second end of the motor shaft comprises a blower-level balanced construction.

In some forms, the blower-level balanced construction comprises mass removal from at least one of four planes each of which extends through a respective one of the pair of spacers, the first impeller, and the second impeller.

In some forms, mass removal from the first impeller and/or the second impeller comprises a notch in a portion of the top shroud between adjacent impeller blades.

In some forms, the first and second impellers are configured such that the first flow and the second flow are substantially similar to one another.

In some forms, the blower only includes the first impeller and the second impeller.

Another aspect of the present technology relates to a blower including a motor configured to drive a motor shaft, the motor shaft having a first end and a second end extending opposite the first end, a first housing including a first inlet, a first impeller connected to the first end of the motor shaft, the first impeller configured to be rotated by the motor shaft to deliver a first flow of air from the first inlet toward an outlet, the first impeller including a plurality of impeller blades and a top shroud and a bottom shroud that define a flow passage therebetween through the first impeller, the top shroud including a center opening which provides an impeller inlet to allow air to enter the flow passage of the first impeller, a second housing including a second inlet, and a second impeller connected to the second end of the motor shaft, the second impeller configured to be rotated by the motor shaft to deliver a second flow of air from the second inlet toward the outlet, the second impeller including a plurality of impeller blades and a top shroud and a bottom shroud that define a flow passage therebetween through the second impeller, the top shroud including a center opening which provides an impeller inlet to allow air to enter the flow passage of the second impeller, wherein the first flow generated by the first impeller and the second flow generated by the second impeller are configured to flow parallel and opposite to one another along at least a portion of their flow path toward the outlet, and the first and second flows are combined at the outlet, and wherein the flow passage of each of the first impeller and the second impeller is configured to produce a mixed flow of air having both an axial and centrifugal component.

In some forms, each of the impeller blades of the first and second impellers is curved along at least a portion of its length such that each of the impeller blades curves away from a direction of rotation of the first and impellers.

In some forms, the plurality of blades for each of the first and second impellers includes primary blades and secondary blades, and wherein each of the primary blades are longer than the secondary blades.

In some forms, the blower only includes the first impeller and the second impeller.

Another aspect of the present technology relates to a two-stage blower configured to produce a parallel flows of air. In some forms, each stage includes a mixed flow impeller.

Another aspect of the present technology relates to a two-stage blower, each stage including a mixed flow impeller. In some forms, one or more of the mixed flow impellers includes anti-fluttering characteristics.

Another aspect of the present technology relates to a blower including one or more mixed flow impellers. In some forms, one or more of the mixed flow impellers includes anti-fluttering characteristics or anti-fluttering means.

Another aspect of the present technology relates to a blower including one or more impellers with anti-fluttering characteristics or anti-fluttering means.

Another aspect of the present technology relates to an impeller including a mixed flow configuration. In some forms, the mixed flow impeller includes anti-fluttering characteristics or anti-fluttering means.

Another aspect of the present technology relates to an impeller including anti-fluttering characteristics or anti-fluttering means.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g., by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

FIG. 1 shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2 shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.1 Portable RPT Device

FIG. 3 is a perspective view of a wearable all-in-one RPT device according to an example of the present technology.

FIG. 4 is a perspective view of the all-in-one RPT device of FIG. 3 worn by a patient according to an example of the present technology.

FIG. 5 is a perspective view of a blower for a wearable all-in-one RPT device in an assembled position according to an example of the present technology.

FIG. 6 is an exploded view of the blower of FIG. 5.

FIG. 7 is an exploded view of the blower of FIG. 5 including a motor.

FIG. 8 is a cross-sectional perspective view of the blower of FIG. 5.

FIG. 9 is a front cross-sectional view of the blower of FIG. 5.

FIG. 10 is cross-sectional view showing flow through the blower of FIG. 5 according to an example of the present technology.

FIG. 11 is a bottom view of an impeller used with the blower of FIG. 5 according to an example of the present technology.

FIG. 12 is a bottom view of an impeller used with the blower of FIG. 5 having a chamfered end to the primary and secondary impeller blades according to an example of the present technology.

FIG. 13 is a bottom perspective view of an impeller used with the blower of FIG. 5 having a cut-out portion according to an example of the present technology.

FIGS. 14, 15, and 16 are a top perspective view of various impellers used with the blower of FIG. 5 having various patterns of serrations according to an example of the present technology.

FIG. 17 is a side view of a stator used with the blower of FIG. 5 according to an example of the present technology.

FIG. 18 is a side view of an alternate stator used with the blower of FIG. 5, illustrating a hollow portion of a stator vane according to an example of the present technology.

FIG. 19 is a cross-sectional view of the stator of FIG. 18 assembled as part of the blower of FIG. 5 according to an example of the present technology.

FIG. 20 is a perspective view of a blower for a wearable RPT device according to an example of the present technology.

FIG. 21 is a perspective view of the blower of FIG. 20 with the housings removed according to an example of the present technology.

FIG. 22 is an exploded view of the blower of FIG. 20.

FIG. 23 is another exploded view of the blower of FIG. 20.

FIG. 24 is another exploded view of the blower of FIG. 20.

FIG. 25 is a cross-sectional view of the blower thru line 25-25 of FIG. 20.

FIG. 26 is cross-sectional view similar to FIG. 25 with components of the motor removed according to an example of the present technology.

FIG. 27A is an enlarged cross-sectional view showing a portion of a stator and elastomeric bearing seat of the blower of FIG. 20 according to an example of the present technology.

FIG. 27B is an enlarged cross-sectional view showing a portion of a stator and elastomeric bearing seat supporting a bearing of the blower of FIG. 20 according to an example of the present technology.

FIG. 28A is an enlarged cross-sectional view showing a portion of a stator and elastomeric bearing seat of the blower of FIG. 20 according to an example of the present technology.

FIG. 28B is an enlarged cross-sectional view showing a portion of a stator and elastomeric bearing seat supporting a bearing of the blower of FIG. 20 according to an example of the present technology.

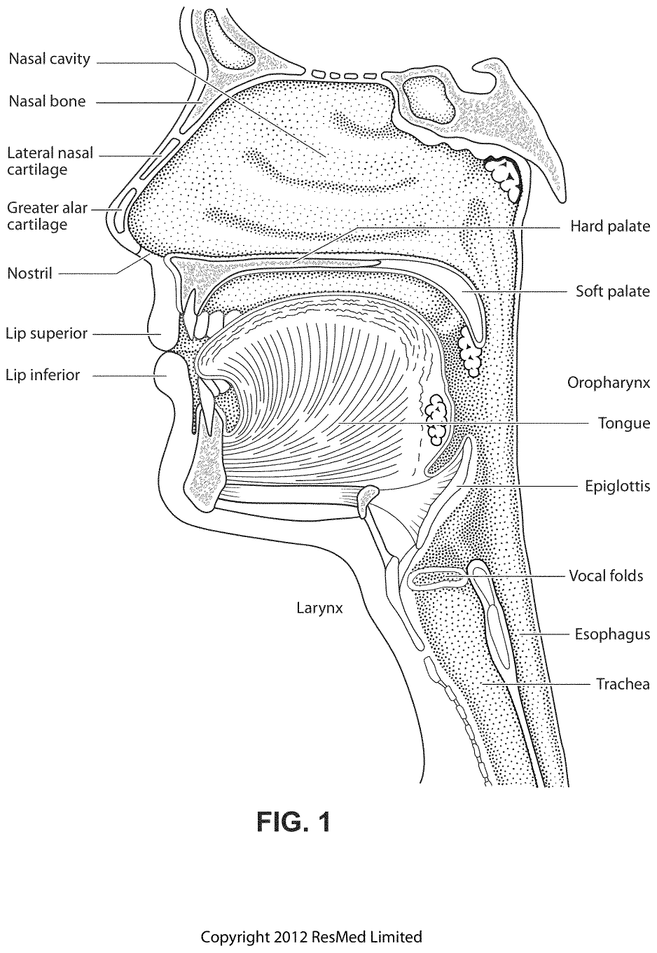

FIGS. 29A, 29B, 29C-1, 29C-2, 29D, 29E-1, 29E-2, 29F, 29G-1, and 29G-2 show an assembly procedure for the blower of FIG. 20 according to an example of the present technology.

FIG. 30 shows a rotor assembly and exemplary balancing planes for balancing at a rotor-level according to an example of the present technology.

FIG. 31 shows assembled portions of a blower and exemplary balancing planes for balancing at a blower-level according to an example of the present technology.

FIG. 32 shows a notched impeller according to an example of the present technology.

### 4.2 Breathing Waveforms

FIG. 33 shows a model typical breath waveform of a person while sleeping.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

In one form, the present technology comprises a system and method for treating a respiratory disorder comprising applying positive pressure to the entrance of the airways of a patient 1000. The system, illustrated in FIGS. 3 and 4 includes a wearable all-in-one RPT device 4000 wherein the patient interface 3000, blower enclosed by enclosure 7050, and battery 7100 are integrated into a positioning and stabilising structure 3300 (e.g., headgear apparatus) and configured to be mounted on the user's head. In alternate forms the patient interface and blower are integrated into a headgear apparatus and a portable battery may be wire linked to power the device. In certain examples, the supply of air at positive pressure is provided to the nasal passages of the patient via an interface including a seal-forming structure 3100 that covers the nares and/or nose of the patient. In certain other examples, the patient interface includes a seal-forming structure that covers the nose and mouth.

The wearable RPT device 4000 in accordance with one aspect of the present technology comprises mechanical, pneumatic, and/or electrical components and is configured to execute one or more algorithms, such as any of the methods, in whole or in part, as described in any one of the following patents or patent applications the contents of which are incorporated herein by reference in their entirety. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 2 cmH2O, or at least 4 cmH2O, or at least 6 cmH2O, or at least 10 cmH2O, or at least 20 cmH2O. In other forms, the RPT device 4000 may maintain a pressure equivalent to an ambient pressure.

### 5.1.1 Pressure Generator

In one form of the present technology, a pressure generator for producing a flow, or a supply, of air at positive pressure is a controllable blower (e.g., blower enclosed by enclosure 7050). For example, the blower may include a brushless DC motor with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The following patents or patent applications are incorporated herein by reference in their entirety: U.S. Pat. Nos. 7,866,944; 8,638,014; 8,636,479; and PCT Patent Application Publication No. WO 2013/020167.

The pressure generator may be under the control of the therapy device controller.

### 5.1.2 Portable/Wearable RPT Device

FIGS. 5-10 illustrate a blower 6000 for the wearable RPT device 4000 according to an example of the present technology.

Blower 6000 may be a device for generating a flow of pressurised air for the RPT device. When used with a patient interface 3000, the blower 6000 may deliver the pressurised air to the patient's airways in order to assist with the patient's breathing. Alternatively or additionally, the blower 6000 may be used for generating a flow of air at an ambient pressure. This could similarly be used to assist with a patient's breathing. Further alternatively or additionally, the blower 6000 could be used to generate a cooling airflow (which may be pressurised or unpressurised). This could make wearing an interface more comfortable for a user (e.g., a patient receiving breathing therapy and/or a person wearing another type of device).

As illustrated in FIG. 5, the blower 6000 may include a substantially cylindrical shape. Specifically, the blower 6000 may include a first or left housing 6010 and a second or right housing 6020, each of which may include a substantially cylindrical shape.

In some forms, the cylindrical shape of the left and right housings 6010, 6020 may provide a smooth surface around an outer perimeter. As will be described below in more detail, the smooth surface may assist with connecting the blower 6000 to another element (e.g., the patient interface 3000).

In certain forms, the blower 6000 may also have a substantially compact design (e.g., as a result of the cylindrical shaped left and right housings 6010, 6020). The compact shape of the blower 6000 may assist with connecting the blower 6000 to another element because the blower 6000 may be capable of fitting into a smaller space. Additionally, the compact design may result in a light-weight blower 6000 (e.g., about 40-50 grams, e.g., 40 grams), which as described in more detail below, may be beneficial for using with the patient interface 3000.

### 5.1.2.1 Housing

As shown in FIG. 6, each of the housings 6010, 6020 may be substantially hollow and include a cavity 6012, 6022 for receiving various elements of the blower 6000. Each cavity 6012, 6022 may have a similar shape (e.g., cylindrical) as the outer shape of the respective housing 6010, 6020.

In some forms, each housing 6010, 6020 may include an opening on either lateral end. For example, the left housing 6010 may include a left cavity opening 6014 and the right housing 6020 may include a right cavity opening 6024. The left and right cavity openings 6014, 6024 may each be substantially circular in shape. The diameter of each cavity opening 6014, 6024 may be similar to the outer diameter of each respective housing 6010, 6020 (e.g., the thickness of each housing 6010, 6020 proximate to the respective cavity opening 6014, 6024 may be relatively small).

In some forms, the diameter of each housing 6010, 6020 may be between about 1 mm and about 50 cm. In some forms, the diameter of each housing 6010, 6020 may be between about 2 mm and about 10 cm. In some forms, the diameter of each housing 6010, 6020 may be between about 1 mm and about 5 cm. In some forms, the diameter of each housing 6010, 6020 may be between about 10 mm and about 50 mm or between about 10 mm and about 30 mm. In some forms, the diameter of each housing 6010, 6020 may be about 20-25 mm, e.g., about 25 mm.

In some forms, each housing 6010, 6020 may include an air inlet 6015, 6025 that fluidly communicates with the respective cavity 6012, 6022. In the illustrated examples, the left air inlet 6015 may be positioned at an opposite end of the left housing 6010 from the left cavity opening 6014. A flow path may therefore exist through the left housing cavity 6012 between the left air inlet 6015 and the left cavity opening 6014 (e.g., see FIG. 10). A similar flow path may exist through the right housing cavity 6022 between the right air inlet 6025 and the right cavity opening 6024 (e.g., see FIG. 10).

In certain forms, each housing 6010, 6020 may include a partially tapered or frustoconical shape. As illustrated in FIGS. 5 to 7, the right housing 6020 may narrow in a direction opposite to the right cavity opening 6024. The diameter of the right air inlet 6025 may therefore be less than the diameter of the right cavity opening 6024. The left housing 6010 may include a similar shape (see e.g., FIGS. 8 and 9), i.e., diameter of the left air inlet 6015 is less than the diameter of the left cavity opening 6014, so that the housings are substantially symmetrical.

In one form, the right housing 6020 may include a substantially flat surface 6027 around the perimeter of the right air inlet 6025. The flat surface 6027 may be substantially perpendicular to an axis along an axial length of the right housing 6020 through a center of the right cavity opening 6024 and the right air inlet 6025. An outer diameter of the flat surface 6027 may be less than the largest outer diameter of the right housing 6020. Although not described separately, the left housing 6010 may include a similar shape (see e.g., FIGS. 8 and 9), i.e., the left housing 6010 may include a substantially flat surface 6017 around the perimeter of the left air inlet 6015.

### 5.1.2.2 Internal Housing Elements

As shown in FIGS. 6 and 7, the blower 6000 may further include left and right stators 6030, 6040, left and right impellers 6050, 6060, and left and right shrouds 6070, 6080. The following description will focus solely on the “left” elements for the sake of brevity, but all of the description is equally applicable to the “right” elements. Additionally, the descriptive terms “left” and “right” refer to the orientation of the figures and could be reversed depending on the orientation of the blower 6000.

### 5.1.2.2.1 Impeller

As illustrated in FIGS. 8 and 9, the left housing 6010 may at least partially house the left stator 6030, the left impeller 6050, and the left shroud 6070. In the illustrated example, the left impeller 6050 is arranged proximate to the left air inlet 6015 and the left stator 6040 includes an end arranged proximate to the left cavity opening 6014. The left shroud 6070 is arranged between the left impeller 6050 and the left stator 6030. The left impeller 6050 and the left shroud 6070 are assembled (e.g., interference or press fit) to the shaft or rotor 7005 of the motor 7000 and cooperate to form a complete double-shrouded impeller for the left side of the blower. Similarly, the right impeller 6060 and the right shroud 6080 are assembled (e.g., interference or press fit) to the shaft or rotor 7005 of the motor 7000 and cooperate to form a complete double-shrouded impeller for the right side of the blower.

In the illustrated example, the left impeller 6050 includes a hub 6052, a top shroud 6051, and a plurality of impeller blades 6054, and the left shroud 6070 includes a hub 6074 and a bottom shroud 6071. The hub 6052, 6074 of the left impeller 6050 and the left shroud 6070 are configured to secure respective parts to the rotor 7005, e.g., via press-fit, interlock, adhesive. The top and bottom shrouds 6051, 6071 are arranged on opposite sides of the impeller blades 6054 and cooperate to define a flow passage 6076 therebetween through the impeller, e.g., to reduce air leakage and improve power efficiency.

In the illustrated example, the left impeller 6050 and the left shroud 6070 are formed separately (e.g., injection molded) from one another (i.e., multi-part construction including 2 separate parts), and then the two parts are assembled separately to the rotor 7005 to form the complete double-shrouded impeller. For example, the left shroud 6070 may be first assembled to an end of the rotor 7005, and then the left impeller 6050 may be subsequently assembled to the end of the rotor 7005 until the hub 6052 of the left impeller 6050 engages the hub 6074 of the left shroud 6070 which consequently allows the impeller blades 6054 of the left impeller 6050 to engage the bottom shroud 6071 provided by the left shroud 6070. In an example, joining the left shroud 6070 directly to the rotor (rather than to the left impeller 6050) to form the complete double-shrouded impeller facilitates manufacturing and eliminates joints (e.g., adherence or weld points between impeller blades 6054 and the bottom shroud 6071), reducing potential failure points to improve the durability of the complete double-shrouded impeller. Additionally, the frusto-conical shape of the left shroud 6070 provides increased stiffness over typical shrouds, reducing the likelihood of vibration. Together, the frusto-conical shape and direct connection between the shroud and rotor shaft both contribute to a robust, durable, and efficient impeller.

In an alternative example, the left impeller 6050 and the left shroud 6070 may be formed separately (e.g., injection molded) from one another, and then lightly fastened to one another (e.g., via an interlock or adhesive) to form a unit before assembly to the rotor 7005. In yet another alternative, the left impeller 6050 and the left shroud 6070 may be integrally formed in one piece.

For the sake of clarity, the elements of the “right” impeller are identified here. For example, the right impeller 6060 includes a hub 6062, a top shroud 6061, and a plurality of impeller blades 6064, and the right shroud 6080 includes a hub 6084 and a bottom shroud 6081. The top and bottom shrouds 6061, 6081 cooperate to define a flow passage 6086 therebetween through the impeller. The description above and below related to the “left” elements is equally applicable to the “right” elements.

In the illustrated example, the blower forms a parallel arrangement because each impeller 6050, 6060 will produce a parallel flow of air through the blower which will eventually combine to achieve a desired flow at the outlet, i.e., each of the left and right sides of the blower generates half of the desired flow rate at the outlet. That is, the first flow generated by the first impeller and the second flow generated by the second impeller are configured to flow parallel and opposite to one another along at least a portion of their flow path toward the outlet, and the first and second flows are combined at the outlet. Preferably, the blower includes only two impellers attached to the rotor 7005.

The parallel stage arrangement may allow the blower 6000 to generate sufficient pressure at typical inspiratory flow rates, while reducing the size of the blower 6000 and reducing its generation of noise. As can be seen in FIGS. 6 to 9, the exemplary blower 6000 includes two total stages (i.e., one impeller 6050, 6060 on each side of the blower), although additional stages may be included (see e.g., U.S. Patent Publication No. 2022/0184335, the entire contents of which is incorporated herein by reference).

In some forms, the impellers 6050, 6060 may be constructed in order to efficiently produce a desired pressure/flow rate at a certain impeller diameter. For example, the impellers 6050, 6060 are operated to provide a sufficient amount of pressure/airflow to the patient in order to be therapeutically effective. However, increasing the speed of the impellers 6050, 6060 (e.g., to adjust flow) may create additional vibrations and/or noise (e.g., noise that follows running speed). Additionally, operation of the blower with a parallel impeller arrangement may produce a fluttering effect whereby one impeller may produce a different flow rate than the other impeller. For example, fluttering may occur when there is an imbalance in flow rates between each side of the blower, i.e., one side of the blower produces a positive flow rate and the other side of the blower produces a negative flow rate. Each side of the blower in the parallel arrangement produces ideally half the desired flow rate, but fluctuating and different flows on each side of the blower cause fluttering, which is noisy and potentially inefficient. Accordingly, the impellers 6050, 6060 according to an example of the present technology are configured (as described below) so that they produce substantially the same flow in use, at both low and high flows. Because flow at each side of the blower substantially matches, fluttering is significantly reduced or eliminated, which improves efficiency of the blower. Also, the impellers 6050, 6060 may be constructed in order to minimize noise output in order to avoid disturbing the patient and/or damaging the blower 6000.

As described below, different characteristics may be applied to the impellers 6050, 6060 in order to achieve desired performance objectives (e.g., pressure/flow) at high efficiency. For example, the impellers 6050, 6060 may include an anti-fluttering characteristic, e.g., curved impeller blades 6054 that are curved away from the direction of rotation. This blade shape is sometimes called backswept or backward-curved. Also, the impellers 6050, 6060 may include a conical shape to provide mixed flow, i.e., axial flow and centrifugal/radial flow. The mixed flow impeller provides desired pressure/flow at a relatively small impeller diameter (e.g., 20-25 mm, e.g., 22 mm).

### 5.1.2.2.1.1 Backswept Blades

As noted above, each impeller 6050, 6060 includes a hub 6052, 6062, and such hubs 6052, 6062 are disposed proximate to a center of the impeller 6050, 6060 (e.g., see FIG. 11). Each hub 6052, 6062 may include a hub opening 6053, 6063 that extends through the hub 6052, 6062 (e.g., a through hole). The plurality of blades 6054, 6064 may extend (e.g., curve) radially outwardly with respect the hub 6052, 6062 (see e.g., FIG. 11).

As illustrated in FIGS. 6 and 10, the blades 6054 may include a curved orientation. For example, the blades 6054 may not extend in a straight-line path between the hub 6052 and the outer perimeter of the left impeller 6050. Instead, they may extend along a curvilinear path in order to form arcuate or curved blades 6054. That is, each of the blades 6054 may provide curved surfaces along its length.

In the illustrated example, each of the blades 6054 curves away from the direction of rotation (direction “Rot” in FIG. 11). This orientation of the blades 6054 may be referred to as back swept because direction of curvature is opposite to the direction of rotation. Each blade 6054 may include a concave surface 6078 and a convex surface 6077 (see FIG. 11), where the convex surface 6077 is a leading surface oriented towards the direction of rotation. For example, the impeller 6050 illustrated in FIG. 11 may rotate in the clockwise direction “Rot” so that the convex surface of each blade 6054 pushes the airflow.

In some forms, the curvature of each blade may relate to a desired blade outlet angle (OA in FIG. 11), which may be selected based on desired performance characteristics (e.g., reduce flutter while providing a desired pressure/flow sufficient for therapy). For context, a straight blade with an axis that extends through the axis of the hub would form a 90° blade outlet angle. Therefore, the blade outlet angle can be decreased by using a curved blade and increasing the curvature of the blade (e.g., forming the blade to be more backswept).

In some forms, the blade outlet angle may be between about 85° and about 1°. In some forms, the blade outlet angle may be between about 70° and about 5°. In some forms, the blade outlet angle may be between about 50° and about 10°. In some forms, the blade outlet angle may be between about 30° and about 15°. In some forms, the blade outlet angle may be about 20°. In some forms, the blade outlet angle may be between about 5° to 50°, e.g., 10°-50°, e.g., 10°-30°, e.g., 10°-20°, e.g., about 10°.

Backswept blades create a falling, monotonic fan curve. So then there is only one possible flow rate, for any given pressure. With a unique flow solution for every pressure, the blower can no longer “flutter” between multiple flow rates, and the fluttering is eliminated.

In some forms, the curvature along each blade 6054 may be substantially uniform (e.g., the radius of curvature may be constant along the length of each blade 6054). The radius of curvature of each blade 6054 may also be the same. The spacing between the adjacent blades 6054 may therefore be substantially constant along the length of the blades 6054.

In other forms, the curvature along at least some of the blades 6054 may vary along its length. For example, a single blade 6054 may have different sections along its length with a larger and smaller radius of curvature. In some forms, the curvature of at least some of the blades 6054 may be different than the curvature on other blades 6054. For example, the radius of curvature (or curvatures) of one blade 6054 may be different from at least one other blade 6054 of the impeller 6050.

In some forms, the impeller blades 6054 may be divided into primary blades 6052a and secondary blades 6052b (see FIG. 11). The primary blades 6052a are longer than the secondary blades 6052b, i.e., the primary blades 6052a extend from the outer perimeter of the impeller to the hub 6052 whereas the secondary blades 6052b are abbreviated and extend from the outer perimeter of the impeller to a position short of the hub 6052. The primary and secondary blades are alternately arranged around the perimeter of the impeller, i.e., each primary blade 6052a may be surrounded on either side by a secondary blade 6052b. In the illustrated example, the impeller includes 14 total blades, i.e., 7 primary blades 6052a and 7 secondary blades 6052b. However, it should be appreciated that more or less blades may be provided, e.g., 8, 10, 12, 16 or more blades. The use of shorter secondary blades uses less space because they do not extend to the hub, which allows an overall smaller size impeller.

In some forms, as shown in FIG. 11, the blades 6054 may be unequally spaced apart from one another along at least a portion of their length. In an example, the primary blade 6052a may be closer to one of the secondary blades 6052b along at least a portion of its length so that it is not equally spaced between the two adjacent secondary blades 6052b.

In certain forms, a secondary blade 6054b is closer to a convex side of one adjacent primary blade 6054a than to the concave surface of the other adjacent primary blade 6054a.

In some forms, the primary blades 6052a and/or the secondary blades 6052b may be unequally spaced apart with respect to themselves. For example, a given primary blade 6052a may be closer to one adjacent primary blade 6052b than to another.

In some forms, the blades 6054 may be spaced apart from one another so that there is substantially equal space between each of the adjacent blades 6054, e.g., spacing between primary and secondary blades may be equal.

The spacing between the blades 6054 (either equally or unequally) may be selected in order to improve efficiency of the impeller 6050. As will be described in more detail below, the top shroud 6051 of the impeller 6050 includes a center opening 6156 (e.g., see FIG. 8) which provides an impeller inlet 6058 to allow air to enter the flow passage 6076, and thus the opening must remain at least partially unobstructed to create the flow path. The use of too many primary blades 6054a (that extend across the opening to the hub) may at least partially obstruct the opening and limit airflow. Also, the use of only primary blades 6054a (without any secondary blades) may also create too large of a space between adjacent blades such that airflow is not produced efficiently by the impeller 6050. Accordingly, the use of both primary blades 6054a and secondary blades 6054b according to an aspect of the present technology may therefore provide efficient spacing between adjacent blades 6054 while also avoiding adverse obstruction of the center opening 6156.

As illustrated in FIGS. 8 and 9, each blade 6054 includes a leading edge 6055 and a trailing edge 6056. It should be noted that the terms “leading edge” and “trailing edge” are to be understood akin to its usage in aeronautics, referring to a portions of a wing, rather than a narrow geometric sense of an “edge”. For example, a “leading edge” may refer to a part of the impeller blade 6054 that generally first contacts the air coming into the left impeller 6050. Similarly, a “trailing edge” may refer to a part of the impeller blade 6054 that generally last contacts the air as it leaves the left impeller 6050.

In the illustrated example, the impeller blades 6054 are sandwiched between the top and bottom shrouds 6051, 6071. Each primary blade 6054a is overlapped by the top shroud 6051 such that its leading edge 6055a is exposed through the opening 6156. That is, the leading edge 6055a extends between the edge of the opening 6156 and the hub 6052 defining the impeller inlet 6058 into the impeller. Each secondary blade 6054b is overlapped by the top shroud 6051 such that its leading edge 6055b is aligned with the edge of the opening 6156, i.e., extends short of the hub. The trailing edge 6056 of the primary and secondary blades 6054a, 6054b are exposed through the impeller outlet 6059 between the outer ends of the top and bottom shrouds 6051, 6071.

According to one aspect of the present technology, the leading edge 6055 and/or the trailing edge 6056 of an impeller blade 6054 may be very thin, such that turbulence and noise is reduced at the inlet and outlet of the left impeller 6050. In an example, the thickness of the leading edge 6055 and/or the trailing edge 6056 of an impeller blade 6054 may be less than about 0.2 mm, e.g., less than about 0.1 mm, such as measured at its thinnest portion, or measured at its outermost portion (i.e., most downstream portion). Furthermore, uniquely to RPT devices, some impeller designs may be such that a seemingly small reduction in a size of the leading (and/or trailing) edge may have a positive effect on the air flow of the left impeller 6050 and efficiency of the RPT device.

In some forms, at least one impeller blade 6054 may comprise one or more serrations 6057, e.g., the leading edge 6055 and/or the trailing edge 6056 may comprise one or more serrations 6057 arranged along the leading edge 6055 and/or the trailing edge 6056. Some examples of potentially suitable arrangements of leading edge and/or trailing edge serrations may be found on U.S. Pat. No. 10,844,876, the contents of which is incorporated herein by reference in its entirety. In the illustrated example, only the leading edges 6055 include one or more serrations 6057, while the trailing edges 6056 are smooth. However, as described above, the trailing edges 6056 may include the serrations in addition to or instead of the leading edges 6055.

In the illustrated example, serrations 6057 are provided along the leading edge 6055a of each primary blade 6054a that is exposed through the opening 6156, e.g., see FIGS. 5 to 10.

As shown in FIGS. 14 to 16, the serrations 6057 may be formed with a variety of different patterns. For example, FIG. 14 illustrates a stepped pattern while FIGS. 15 and 16 illustrate a curved pattern. Each of these patterns, and other similar unillustrated patterns are configured and arranged to break up turbulence of air entering the flow passage 6076 in order to assist with noise control, i.e., reduce turbulent noise.

In some forms, the curvature of the blades 6054 described above may provide a curved surface 6158 toward the respective trailing edge 6056 (e.g., see FIG. 12). This may assist in providing a substantially smooth flow path in order to reduce turbulence, and thus noise.

As shown in FIG. 9, at least some of the leading edges 6055 may be substantially perpendicular to the hub 6052 (e.g., not including an incline). In other examples, at least one leading edge 6055 of a blade 6054 may be inclined, e.g., by an angle greater than 45 degrees, with respect to an axis of the hub 6052.

With continued reference to FIG. 9, the leading edges 6055 (and any serrations 6057) may be exposed through the air inlet 6015 of the housing 6010. In the illustrated example, the leading edges 6055 may be recessed from the flat surface 6017 of the housing 6010. However, in other examples, the leading edges 6055 may be co-planar with the flat surface 6017 or extend through the air inlet 6015 and outside of the housing 6010.

In some forms, the backswept shape of the blades 6054 may produce a fan curve (e.g., a relationship between flow rate and pressure) that includes a gradient, which may produce only one solution for a given impeller 6050. In other words, the backswept blades 6054 may produce a single operating point and therefore reduce or eliminate fluttering (e.g., where multiple operating points exist thereby causing different outputs by the fan at different points).

In the illustrated example, each side of the blower includes a respective impeller 6050, 6060 with backswept blades which produce substantially similar flow, even at low flows, which minimizes or eliminates fluttering or flow imbalance. By minimizing or eliminating flutter, the blower can perform at a higher efficiency while delivering desired pressure/flow.

In some forms, at least some of the blades 6054 may be formed with chamfered or rounded ends. For example, one or both of the primary blades 6054a and the secondary blades 6054b may include chamfered ends. The chamfered ends may act similar to the serrations 6057 described above. In other words, the chamfered ends may assist in breaking up turbulent flow in order to limit noise output.

In some forms, at least some of the blades 6054 may be formed with cut-outs, e.g., one or more cut-outs provided along the leading edge 6055a of each primary blade 6054a as shown in FIG. 14. For example, one or both of the primary blades 6054a and the secondary blades 6054b may include cut-outs. In an examples, the cut-outs may be formed on the convex side of the blades 6054. The cut-outs may assist in reducing stress on the blades 6054 during use.

### 5.1.2.2.1.2 Mixed Flow Impeller

As described above, each impeller 6050, 6060 and respective shroud 6070, 6080 cooperate to form a complete double-shrouded impeller for the respective side of the blower. In the illustrated example, each complete double-shrouded impeller is in the form of a mixed flow impeller structured to generate mixed flow, i.e., axial flow and centrifugal/radial flow. In this regard, each of the top shroud 6051 and the bottom shroud 6071 is substantially non-planar, e.g., each of the top shroud 6051 and the bottom shroud 6071 comprises a frusto-conical shape. For example, each of the top shroud 6051 and the bottom shroud 6071 may taper in the radial direction with respect to the axial direction of the impeller, which arrangement defines a flow passage 6076 through the impeller with both axial and radial components.

In some forms, the top shroud 6051 defines an outer surface of the impeller 6050 having an inclined shape (e.g., as viewed in cross section). In the illustrated example, at least a portion of the housing adjacent the impeller 6050 may include a frustoconical shape so as to conform to the frustoconical shape of the impeller. The outer surface of the top shroud 6051 may face toward the outer end of the blower 6000. In an example, at least a portion of the outer surface of the top shroud 6051 may include a curvature. The outer surface of the top shroud 6051 may be opposite of the blades 6054, which may face toward a center of the blower 6000. As shown in FIG. 9, the incline of the impeller 6050 may be substantially the same as the incline of at least a portion of the housing 6010 adjacent the impeller so that the spacing or gap between the outer surface of the top shroud 6051 and the housing remain substantially constant.

In some forms, at least a portion of the blades 6054 may be disposed on a concave surface of the bottom shroud 6071 and/or a convex surface of the top shroud 6051. The concave and convex surfaces are illustrated as being substantially smooth.

As noted above, each primary blade 6054a extends from the hub 6052 to the outer perimeter of the impeller, and each secondary blade 6054b extends from the edge of the opening 6156 to the outer perimeter of the impeller. That is, in some forms as shown in FIG. 11, at least some of the blades 6054 may extend entirely between the hub 6052 and the outer perimeter of the left impeller 6050, and some of the blades 6054 may extend to the outer perimeter of the left impeller 6050 but may not extend completely to the hub 6052. For example, the blades 6054 may be arranged so that every other blade 6054 is connected to the hub 6052 (i.e., the primary blades 6054a), and each blade 6054 not connected to the hub 6052 (i.e., the secondary blades 6054b) is between two blades 6054 that are. As noted above, the use of both longer primary blades and shorter secondary blades efficiently utilizes space to enable a smaller size impeller. For example, the secondary blades guide the air better near the outer part of the impeller, without reducing the cross-sectional area too much at the inlet.

In the illustrated example, the top shroud 6051 forms an opening 6156 that is disposed between hub 6052 and at least some of the blades 6054. For example, the blades 6054 that do not extend to the hub 6052 (e.g., secondary blades 6054b) may extend to an outer diameter of the opening 6156 and may be spaced apart from the hub 6052 by the width of the opening 6156. The opening 6156 may thus be considered radially outside of the hub 6052. Additionally, the blades 6054 that do extend to the hub 6052 (e.g., primary blades 6054a) may extend across the opening 6156, which exposes the leading edge 6055a of the primary blade 6054a to air entering the impeller. The primary blades 6054a may divide the opening 6156 into a plurality of discrete openings, each bounded by two primary blades 6054a that extend to the hub 6052.

In the illustrated example, the opening 6156 provides an impeller inlet 6058 to allow air to enter the flow passage 6076. For example, air in the ambient environment may enter the system through the opening 6156 in order to flow into the flow passages and into contact with the blades 6054. The opening 6156 may be oriented so that its axis is substantially coaxial to the motor shaft.

In some forms, there may be an even number of total blades 6054, with an equivalent number of blades 6054 that are connected to the hub 6052 as are not connected to the hub 6052. In some forms, there may be at least three of each type of blade 6054. In some forms, there may be at least five of each type of blade 6054. In some forms, there may be at least seven of each type of blade 6054. In some forms, there may be at least ten of each type of blade 6054. In some forms, there may be at least twenty of each type of blade 6054.

In other examples, some blades 6054 may extend from the hub 6052 and not reach the outer perimeter of the left impeller 6050. In other examples, some blades 6054 may be spaced apart from both the hub 6052 and the outer perimeter of the left impeller 6050.

As noted above, the impeller 6050 and shroud 6070 cooperate to form a hybrid or mixed flow impeller (e.g., at least part axial and part centrifugal/radial) as a result of its shape. For example, the opening 6156 may form at least part of an axial flow path for airflow to enter the impeller. In addition, the conical or frustoconical shape of the flow passage 6076 formed by the top and bottom shrouds 6051, 6071 forms at least part axial and radial flow path for airflow travelling towards the impeller outlet. For example, after passing through the opening 6156, the airflow may acquire a radial component of travel.

The mixed flow impeller 6050 may enable the blower 6000 to achieve desired pressure/flow at a smaller size, e.g., a smaller size to provide a more compact and lighter blower 6000, while still achieving a sufficient pressure for therapeutic effectiveness.

### 5.1.2.2.2 Shroud

As shown in FIGS. 6 to 9, the left shroud 6070 is configured to be positioned proximate to the left impeller 6050 to form the complete double-shrouded impeller within the left housing 6010 of the blower. In the illustrated example, the left shroud 6070 may be positioned adjacent to the blades 6054 of the impeller 6050. The left shroud 6070 may therefore be known as the bottom shroud.

In the illustrated example, the bottom shroud 7071 formed by the left shroud 6070 may include an inclined surface. In some forms, at least a portion of this surface may be curved, e.g., so that it is similar to the shape of the housing 6010, and to the shape of the impeller 6050.

In some forms, the inclined shape of the left shroud 6070 may be different than the inclined shape of the left impeller 6050. For example, as illustrated in FIG. 9, the top shroud 6051 of the left impeller 6050 may have a steeper incline than the bottom shroud 6071 of the left shroud 6070. The distance between the shrouds of the left impeller 6050 and the left shroud 6070 may decrease toward the outer edge of both the left impeller 6050 and the left shroud 6070. That is, the flow passage 6076 formed between the top and bottom shrouds 6051, 6071 may narrow or taper from the impeller inlet 6058 to the impeller outlet 6059.

In other examples, the bottom shroud 6071 of the left shroud 6070 may have a steeper incline (such that the distance between the top and bottom shrouds increases) or they may have the same incline, so that the distance remains substantially the same.

As shown in FIG. 6, the surfaces of the left shroud 6070 and the right shroud 6080 may be smooth (e.g., lack serrations and/or blades). As shown in FIGS. 8 and 9, the smooth surface of the shrouds 6070, 6080 may allow the respective blades 6054 to contact substantially flush against the surface of the respective shroud 6070, 6080.

As shown in FIGS. 8 and 9, the outer edge of the left shroud 6070 forms a diameter that is substantially similar to the outer diameter of the left impeller 6050. For example, the outer diameters of the left shroud 6070 and the left impeller 6050 may be equal. In an alternative example, such outer diameters may be different than one another, e.g., left impeller 6050 forms a larger outer diameter than the left shroud.

In some forms, the impellers each include an outer diameter between about 20 mm to about 30 mm, e.g., 20-25 mm, e.g., 22 mm.

Returning to FIG. 6, the right shroud 6080 (and similarly the left shroud 6070, although not illustrated in FIG. 6) may include a shroud clip 6082, which may assist in connecting and/or aligning the respective shroud 6070, 6080 to the respective impeller 6050, 6060. The shroud clip 6082 may be disposed around the perimeter of the shroud hub 6084. In the illustrated example, the shroud clip 6082 may be formed from a series of discontinuous elements (e.g., equally spaced elements).

As shown in FIGS. 8 and 9, the shroud clips 6072, 6082 may connect to the outer surface of the respective hub 6052, 6062. In some forms, this may be a removable connection (e.g., a snap-fit, friction fit, press fit, etc.) and may allow the respective shroud 6070, 6080 to be removed from the respective impeller 6050, 6060. In this form, the respective shroud 6070, 6080 and the respective impeller 6050, 6060 may not be bonded or connected together, e.g., before or after assembly to the rotor.

In other forms, the shroud clips 6072, 6082 may provide a permanent connection between the respective shroud 6070, 6080 and impeller 6050, 6060. For example, the respective shroud 6070, 6080 and the respective impeller 6050, 6060 may each be formed as a single piece (e.g., using injection molding, 3-D printing, etc.).

When connected, the left impeller 6050 and the left shroud 6070 cooperate to form a flow passage 6076 therebetween through the impeller 6050 through which a flow of air may travel. The flow passage 6076 extends from the impeller inlet 6058 at an inner portion of the impeller 6050 to an impeller outlet 6059 at an outer portion of the impeller 6059. The impeller inlet 6058 may be formed between adjacent leading edges 6055, and the impeller outlet 6059 may be formed between adjacent trailing edges 6056. The flow passage 6076 may include a plurality of channels, each channel formed at least partly by the left impeller 6050 and the left shroud 6070 and impeller blades 6054.

In some forms, the arrangement of the impeller 6050 and the shroud 6070 to form the flow passage 6076 may assist in improving efficiency. For example, the left shroud 6070 may limit leakage of air driven by the left impeller 6050, and thus more efficiently direct air toward the blower outlet 6098 (and to the patient).

As described above, the flow passages 6076 formed between the left impeller 6050 and the left shroud 6070 are structured to narrow from the impeller inlet 6058 to the impeller outlet 6059, i.e., the spacing or distance between the impeller 6050 and shrouds 6070 lessens or tapers from the impeller inlet 6058 to the impeller outlet 6059.

This may be the result of the different inclines in the left impeller 6050 and the left shroud 6070. As a result of the steeper incline in the left impeller 6050 (e.g., as compared to the left shroud 6070), the surfaces of the left impeller 6050 and the left shroud 6070 may become closer together along the length of the flow passage 6076 in the direction of the impeller outlet 6059.

When connected, the shroud hub 6074 of the left shroud 6070 may be aligned with the left hub 6052 of the left impeller 6050. In other words, centers of each hub 6052, 6074 may be aligned along a common axis. As will be described in more detail below, the common axis may be a rotor axis along which a drive shaft may extend and extend through both hubs 6052, 6074.

For the sake of clarity, the “right” elements are identified here. The blower 6000 also includes a right shroud 6080, a shroud clip 6082, a shroud hub 6084, and a flow path 6086. Description related to the “left” elements is equally applicable to the “right” elements.

### 5.1.2.2.3 Stator

In the illustrated example, the blower 6000 includes left and right stators 6030, 6040 that that are arranged adjacent to corresponding impellers 6050, 6060 and shrouds 6070, 6080. FIGS. 6 to 9 depict features of an example first or left stator 6030, which together with impeller 6050 and shroud 6070 form the first compression stage 6090. Likewise, the right stator 6040, together with impeller 6060 and shroud 6080 form a second compression stage 6095. Accordingly, the blower includes two stages arranged in parallel configured to efficiently generate sufficient pressure/flow at a reduced size.

The left stator 6030 may include a plurality of left stator vanes 6032 to direct the flow of air from the first impeller 6050 along the outer surface of the left stator 6030. In some forms, this may reduce the velocity of the flow of air from the first impeller 6050 and/or increase the pressure of the flow of air from the first impeller 6050.

The illustrated example may include four stator vanes 6032, although any number of stator vanes 6032 (e.g., two, three, five, six, etc.) may be used on the stator 6030.

In some forms, the number of stator vanes 6032 may be selected for manufacturing and/or performance parameters. For example, the stator 6030 may be manufactured using injection molding process. Limiting the number of stator vanes 6032 may improve the manufacturability of the stator 6030 as a whole. Thus, it may be preferable to minimize the number of stator vanes 6032, while ensuring that the total number of stator vanes 6032 is sufficient for achieving therapeutically effective pressure.

In some forms, at least one of the stator vanes 6032 (e.g., all of the stator vanes 6032) may include a hollow or partially hollow section. As illustrated in FIGS. 18 and 19, a central portion 6031 of each stator vane 6032 may be hollow, or recessed from the surrounding outer portions of respective stator vane 6032. In other words, an interior portion of the stator vane (e.g., the central portion 6031) may include an opening bounded at least partially by the stator vane 6032 and the surface of the stator 6030.

In certain forms, the hollow section of the central portion 6031 may improve manufacturability of the stator 6032 by making the stator 6032 easier to mold.

In the illustrated example, the left stator 6030 may be at least partially received within the left housing 6010 along with the left impeller 6050 and the left shroud 6070.

As described above, the left stator 6030 may be positioned downstream from both the left impeller 6050 and the left shroud 6070. The illustrated examples of FIGS. 8 and 9 show that the left impeller 6050 is positioned further upstream from both the left shroud 6070 and the left stator 6030. However, any orientation of these three elements may be contemplated.

The left stator 6030 may include a top ring 6034, a base ring 6036, and a plurality of left stator vanes 6032 between the top ring 6034 and the base ring 6036. The left stator vanes 6032 may direct the flow of air from the second impeller 6050 to the blower outlet 6098 in a radial and axial direction.

Each of the plurality of left stator vanes 6032 may extend substantially between the top ring 6034 and the base ring 6036 (see e.g., FIG. 17). The left stator vanes 6032 may also include a substantially helical shape. In the illustrated example of FIG. 6, each stator vane 6032 may extend less than 360°, although in other examples, at least one stator vane 6032 may extend greater than 360°.

In some forms, each stator vane 6032 may include a leading edge 6033 and a trailing edge or outlet rib 6037 on an opposite end of the stator vane 6032 from the leading edge 6033. In the illustrated example, the leading edge 6033 may be positioned proximate to the top ring 6034 and the trailing edge 6037 may be positioned proximate to the base ring 6036. In use, the leading edge 6033 may be considered upstream from the trailing edge 6037 in relation to the direction of flow only the stator 6030.

As shown in FIGS. 17 and 18, the leading edge 6033 may be thinner than the trailing edge 6037. The thinner leading edge 6033 may allow airflow exiting the impeller 6050 to more easily flow around and along the stator 6030. The trailing edge 6037 may be thicker than the leading edge 6033 in order to assist in redirecting the airflow.

As shown in FIGS. 17 and 18, the outlet ribs 6037 may be formed with a curvature or bend 6035 to smoothly guide air to the outlet 6098. The bend 6035 may be formed with approximately a 90° angle, although any similar angle (e.g., greater than about 60°) may be used. The bend 6035 may orient an end of each outlet rib 6037 in the radial direction as opposed to the axial direction like much of the remainder of the stator vane 6032.

In some forms, the outlet rib 6037 may be formed at a base of each stator vane 6032 and may join the respective stator vane 6032 to the base ring 6036. The outlet ribs 6037 may project further in a radial direction than the remainder of the stator vanes 6032.

In certain forms, the outlet ribs 6037 may also extend substantially up to the outer perimeter of the base ring 6036. For example, the outlet ribs may be flush with the outer perimeter of the base ring 6036.

As shown in FIGS. 18 and 19, the opening in the central portion 6031 may be formed proximate to the outlet rib 6037. For example, the central portion 6031 may extend substantially to the base ring 6036.

In certain forms, the opening in the central portion 6031 may not extend to an upstream end of the stator vane 6032 (e.g., the leading edge 6033) proximate to the top ring 6034.

In some forms, the left stator vanes 6032 reduce the velocity of the flow of air from the left impeller 6050, and increase the pressure of the flow of air from the left impeller 6050. This may be accomplished with or without the opening in the central portion 6031. Each of the left stator vanes 6032 may have a constant depth D in a radial direction and an increasing width W in a circumferential direction from the top ring 6034 to the base ring 6036.

The stator vanes 6032 may also assist in redirecting the flow of pressurized air. As described above, the stator vanes 6032 assist in directing air through the blower outlet 6098. One aspect of this is radially directing that air so that it can turn and enter the blower outlet 6098. The stator vanes 6032 may be thicker proximate to the outlet rib 6037 in order to provide a structure sufficient to redirect the air. A thinner structure (e.g., similar in size to the leading edge 6033) may not efficiently redirect the air through the blower outlet 6098.

In some forms, the top ring 6034 may include an inclined shape (e.g., as viewed in cross section) so that it can conform to the frustoconical shape of the impeller. The top ring 6034 may include a curvature. As shown in FIG. 9, the incline of the top ring 6034 may be substantially the same as the incline of the left shroud 6070 so that the spacing between the top ring 6034 and the left shroud 6070 remain substantially constant. However, other examples may include different inclines and/or varying inclines on the top ring 6034 and/or the left shroud 6070 so that the spacing between the two elements is not substantially constant.

With continued reference to FIGS. 8 and 9, the left stator 6030 may include an outer wall 6038. The left stator vanes 6032 may extend from the outer wall 6038. In the illustrated form, the left stator vanes 6032 may be integrally formed with the outer wall 6038 (e.g., formed during the same moulding process).

In some forms, the outer wall 6038 is continuous between the top ring 6034 and the base ring 6036. For example, the wall 6038 may not contain any openings or other means of ingress or egress.

As shown in FIGS. 8 and 9, certain forms of the left stator 6030 may be hollow. For example, the outer wall 6038 may form an inner perimeter and an outer perimeter of the left stator 6030 (e.g., across the thickness of the outer wall 6038), but may leave an open space within the outer wall 6038.

In some forms, the top ring 6034 may include a hub 6039 with an opening that communicates with the hollow interior formed by the outer wall 6038. In the illustrated example, the hub 6039 may be approximately centered on the top ring 6034 in order to align with the hubs 6052, 6074 of the impeller 6050 and the shroud 6070 respectively. In other words, a common axis may extend through a center of each of the hubs 6039, 6052, 6072.

In some forms, the inner diameter of the hub 6039 of the stator 6030 may be larger than the outer diameter of the hub 6074 of the shroud 6070, e.g., to allow at least a portion of the hub 6074 to extend therethrough.

For the sake of clarity, the “right” elements are identified here. The blower 6000 also includes a right stator 6040, a stator vane 6042, a top ring 6044, a base ring 6046, an outlet rib 6047, an outer wall 6048, and a hub 6049. Description related to the “left” elements is equally applicable to the “right” elements.

### 5.1.2.3 Use of Blower

As described above, the blower 6000 may be assembled so that the elements are at least partially within the respective housing 6010, 6020. In some forms, at least the left and right stators 6030, 6040 may be at least partially positioned outside of the respective housing 6010, 6020. For example, the left housing 6010 may extend along the length of the outer wall 6038 up to the base ring 6036.

In some forms, the base ring 6036 may act as a stop and limit further axial translation of the stator 6030 into the left housing 6010. In this position, the base ring 6036 may remain outside of the housing 6010, while the left stator vanes 6032 remain substantially within the housing 6010. The right housing 6020 and the right stator 6040 may be similarly oriented so that the base ring 6046 extends out of the right housing 6020.

In some forms, the left and right sides may be connected by positioning the left base ring 6036 proximate to the right base ring 6046. As shown in FIG. 9, the base rings 6036, 6046 of the stators 6030, 6040 may contact one another so that the ends of the housings 6010, 6020 are spaced apart from one another. This spacing may create the blower outlet 6098.

As shown in FIGS. 6 to 9, the left and right inlets 6015, 6025 may be generally perpendicular to the blower outlet 6098 which is formed proximate to the middle of the blower 6000 in a circumferential direction.

In certain forms, each housing 6010, 6020 may contact the respective outlet rib 6037, 6047, in use. For example, the outlet ribs 6037 may act as a seat for the left housing 6010 and the outlet ribs 6047 may act as a seat for the right housing 6020. In other words, each housing 6010, 6020 may contact an upper surface (e.g., facing toward the respective top ring 6034, 6044). The end of each housing 6010, 6020 may therefore be spaced apart from the end of the base ring 6036, 6046.

Although the base rings 6036, 6046 may be flush with one another as described above, the housings 6010, 6020 may not be flush with one another as a result of their contact with the respective outlet ribs 6037, 6047. However, the outlet ribs 6037, 6047 are positioned at a base of a respective stator vane 6032, 6042 and are therefore spaced apart from one another. Portions of the blower outlet 6098 are located between each adjacent pair of outlet ribs 6037 and outlet ribs 6047. In other words, as shown in FIG. 8, the locations along the blower 6000 (e.g., along its perimeter) that lack the ribs 6037, 6047, and therefore include a more depressed spacing between the housings 6010, 6020 are portions of the blower outlet 6098.

In some forms, the left housing 6010 may be at least partially spaced apart from the outer wall 6038. For example, the inner diameter of the left housing 6010 may be approximately equal to the outermost dimension of the left stator vanes 6032. Thus, the left stator vanes 6032 may be in contact with (or closely spaced apart from) the left housing 6010, while the remainder of the surface of the outer wall 6038 may be further spaced apart. This may form a flow path along the outer wall 6038 and between the adjacent stator vanes 6032.

In some forms, the air may flow through this space as it is directed along the stator vanes 6032. The outlet ribs 6037 at the base of the stator vanes 6032 may help to direct the airflow toward the blower outlet 6098. In other words, the outlet ribs 6037 are solid and limit the continued airflow at that discrete location. The outlet ribs 6037 therefore direct the airflow to where there is an opening (e.g., the blower outlet 6098).

As shown in FIGS. 18 and 19, the bend 6035 may orient the outlet rib 6037 from a generally axial direction to a generally radial direction.

This airflow may be generated by a motor 7000 (see FIG. 7). The motor 7000 may have a single shaft 7005 protruding from each end thereof to drive corresponding impellers 6050, 6060. In some forms, the motor 7000 may be a brushless DC motor.

The flow of air along each stator 6030, 6040 may be generally along the axial direction. As a result of the impellers 6050, 6060 formed on either side of the motor 7000, parallel streams of air may move toward the center of the blower 6000. In other words, the stream of air produced by one impeller 6050, 6060 is directed toward the stream produced by the other impeller 6060, 6050.

As described above, one impeller may be located on either side of the motor 7000 (e.g., a left impeller 6050 and a right impeller 6060). The impellers 6050, 6060 may therefore be positioned in parallel. A parallel fan arrangement combines flow from each side of the blower to generate a desired flow at the outlet located at the center of the blower 6000.

Additionally, as described above, each impeller 6050, 6060 may be a hybrid impeller that may produce a mixed flow of air (e.g., part axial and part centrifugal airflow). The mixed flow impeller along with the parallel fan arrangement allows a smaller overall size with sufficient flow/pressure to be therapeutically effective.

As each impeller at opposing ends of the motor are configured to generate a flow of gas in opposing directions to each other, while being driven by the same shaft 7005, each opposing impeller may comprise a mirrored geometry. For example, the blades of the impeller 6050 located at a first end of the shaft of the motor may comprise a mirrored geometry to the blades of the impeller 6060. Since both ends of the shaft 7005 will be rotating in the same direction when the motor is operating, the blades of the impellers 6050, 6060 at each respective end of the shaft may configured to be swept or curved backward relative to the shaft's rotational direction.

As shown in FIGS. 8 and 9, the hollow space within the outer walls 6038, 6048 of the stators 6030, 6040 may form a space to receive the motor 7000. The space may have a substantially cylindrical shape, which may correspond to the shape of the motor 7000.

As described above, a common axis RA may extend through a center of each of the hubs 6039, 6052, 6074 (as well as hubs 6049, 6062, 6082). When positioned within the space created by the outer walls 6038, 6048, a center of the motor 7000 may be aligned with the axis RA. The shaft 7005 may protrude from the motor 7000 along the axis RA so that the shaft 7005 may extend through the hubs 6039, 6052, 6074 (as well as hubs 6049, 6062, 6082). Providing power to the motor 7000 causes the shaft to rotate 7005, which in turn causes the impellers 6050, 6060 and shrouds 6070, 6080 to rotate. The rotation of the impellers 6050, 6060 and shrouds 6070, 6080 causes the airflow, which as described above, is directed along the stator vanes 6032, 6042 and out of the blower outlet 6098, e.g., see FIG. 10.

In some forms, the shaft 7005 may have a length between about 1 mm and about 200 mm. In some forms, the shaft 7005 may have a length between about 2 mm and about 100 mm. In some forms, the shaft 7005 may have a length between about 5 mm and about 75 mm. In some forms, the shaft 7005 may have a length between about 10 mm and about 50 mm. In some forms, the shaft 7005 may have a length between about 40 mm and about 45 mm. In some forms, the shaft 7005 may have a length between about 40-60 mm, e.g., 40-50 mm, e.g., 40-45 mm, e.g., about 44 mm.

In some forms, the shaft 7005 may have an outer diameter of between about 0.01 mm and about 10 mm. In some forms, the shaft 7005 may have an outer diameter of between about 0.1 mm and about 5 mm. In some forms, the shaft 7005 may have an outer diameter of between about 0.5 mm and about 3 mm. In some forms, the shaft 7005 may have an outer diameter of between about 1 mm and about 5 mm. In some forms, the shaft 7005 may have an outer diameter of between about 2-3 mm, e.g., 3 mm.

In some forms, increasing the diameter of each housing 6010, 6020 and associated impellers and stators described above may allow for a shorter length of the combined housings 6010, 6020 while maintaining a similar volume. The shorter length of the combined housings 6010, 6020 may therefore allow for a shorter shaft 7005 (e.g., as compared to housings with smaller diameters and/or multiple impellers in series). The shaft 7005 may also be formed with an increased diameter (e.g., as compared to housings with smaller diameters). The shorter and thicker shaft 7005 may have an increased stiffness, which may improve performance.

In some forms, the blower may have a total length along its longitudinal axis between about 30-80 mm, e.g., 40-60 mm, e.g., 40-50 mm, e.g., about 45 mm. In some forms, the blower may have an outside diameter between about 15-50 mm, e.g., 20-30, e.g., 20-25 mm, e.g., about 25 mm.

In some forms, the ratio between the outside diameter of the blower to the length of the blower may be about 0.3 to about 0.7, or about 0.5 to about 0.6, e.g., about 0.54 to 0.56. Moreover, the length of the blower may be about 2 to about 5 times the diameter of the blower and/or impeller.

By shortening and stiffening the shaft 7005, the shaft 7005 may behave less like a flexible member, and may therefore exhibit a decrease in vibrations that contribute to flexion. At high speeds necessary to achieve a therapeutically effective pressure, a stiffer motor shaft 7005 may assist in limiting noise of the shaft 7005, e.g., noise that follows running speed.

As shown in FIGS. 3 and 4, some forms of this blower 6000 may be used with a patient interface 3000, e.g., where the blower is mounted or communicated with the plenum chamber of the patient interface. For example, the blower 6000 may be directly mounted onto the plenum chamber of the patient interface 3000 so that the patient supports the blower on their face, instead of in a separate device.

As shown in FIGS. 3 and 4, the patient interface 3000 may include an enclosure 7050 that may enclose the blower 6000 (not shown in this view), such as the examples disclosed elsewhere herein. The enclosure 7050 may provide sound deadening, e.g., via a muffler and/or acoustic foam. The enclosure 7050 may also include openings (not shown in this view) to allow air to enter the enclosure 7050 to reach the motor 7000 and to allow air to escape via the blower outlet 6098, which may also be at least partly enclosed by the enclosure 7050. The enclosure 7050 may assist in muffling the noise to limit disturbances caused to the patient and/or the bed partner.

In use, the motor 7000 may drive the impellers 6050, 6060 to draw air into the blower through the respective inlets 6015, 6025, which directs the air to follow a generally axial path to the outlet 6098. Upon reaching the respective impeller 6050, 6060, the flow becomes mixed as a result of the impeller shape (e.g., conical or frustoconical) described above. After exiting the respective impeller 6050, 6060, the parallel streams of air flow along the respective stator 6030, 6040 toward a center of the blower. This flow is generally axial, although there may be some radial components as a result of the stator vanes. Finally, the outlet ribs 6037, 6047 assist in directing the two flows of air through the blower outlet 6098. This changes the flow to having a mostly radial direction in order to exit through the blower outlet disposed circumferentially around the blower 6000.

As shown in FIGS. 8 and 9, the blower outlet 6098 may be disposed around the perimeter of the blower 6000. Thus, airflow is output in multiple directions. The enclosure 7050 may help to direct the air by including only a single outlet directed toward the plenum chamber of the patient interface 3000 in order to limit the pressurised air from escaping to ambient.

In use, the blower 6000 may provide pressurised breathable gas to the plenum chamber of the patient interface 3000 so that the patient can inhale the gas. In some forms, the blower 6000 may be controllable (e.g., by an actuator located on the patient interface 3000, by a downloaded program, and/or by a remote control device (e.g., a cell phone or computer)). Controlling the blower 6000 may adjust the speed of the motor 7000 and/or a duration of operation.

FIGS. 20 to 32 show a blower 6000 for a wearable RPT device 4000 according to another example of the present technology. These figures illustrate further aspects of the motor 7000 and exemplary support by the stators 6030, 6040 according to an example of the present technology.

In the illustrated example, the motor 7000 may be a brushless DC motor including shaft or rotor 7005, a permanent magnet 7010 mounted on the rotor 7005, and a stator assembly 7015 that surrounds the rotor 7005 and the magnet 7010 thereof. As illustrated, the stator assembly 7015 includes windings 7016 and a lamination stack 7018 provided to the windings 7016. In some forms, e.g., as shown in FIGS. 22 and 25, each end of the windings 7016 may include a radially outwardly extending end turn 7017, e.g., to retain the windings 7016 in position.

The rotor 7005 is rotatably supported by a pair of bearings 7021, 7022 that are retained or housed by the stator 6040. The bearings 7021, 7022 may be any suitable type as known in the art, e.g., rolling element bearings, fluid bearings (air or liquid), sleeve bearings, or other suitable types. In use, an electronic controller controls operation of the stator assembly 7015 to control spinning movement of the rotor 7005 and hence the impellers 6050, 6060.

In an example, each of the bearings 7021, 7022 is in the form of a rolling element bearing including an inner race, an outer race, and ball bearings between the inner and outer races. The inner and outer races provide surfaces upon which the ball bearings run in use. In an example, the ball bearings may be formed of ceramic material, e.g., to enhance bearing life. For example, the ceramic ball bearings are lightweight and provide lower friction to reduce stress/heat on the inner and outer races during high running speeds of the rotor 7005.

In the illustrated example, the stator 6040 comprises end wall 7210, a bearing tube 7220, and a cylindrical outer wall 7230 arranged radially outwardly of the bearing tube 7220. In the illustrated embodiment, the end wall 7210, bearing tube 7220, and outer wall 7230 may be integrally formed (e.g., injection molded of plastic material) as a one-piece structure. As described above, a plurality of stator vanes 7232 (e.g., four stator vanes) are provided to the outer wall 7230 to direct the flow of air from the impeller 6060 along the stator 6040 to the blower outlet 6098. In the illustrated example, as described above, a central portion 7233 of each stator vane 7232 may be hollow, or recessed from the surrounding outer portions of the respective stator vane 7232.

The stator assembly 7015 is provided to a central portion of the bearing tube 7220 along an exterior surface thereof. The bearings 7021, 7022 are provided to respective end portions of the bearing tube 7220 along an interior thereof. The bearings 7021, 7022 support the rotor 7005 and magnet 7010 within the interior of the bearing tube 7220. The bearing tube 7220 comprises at least a portion that is sufficiently magnetically transparent to allow a magnetic field to pass through it, which allows the stator assembly 7015 along its exterior surface to act on the magnet 7010 positioned within the bearing tube 7220.

In the illustrated example, e.g., see FIGS. 25 to 28B, an elastomeric bearing seat or sleeve 7240 (e.g., comprising an elastomeric material, e.g., thermo-plastic elastomer (TPE), thermo-plastic polyurethane (TPU), thermo-plastic vulcanizates (TPV), and liquid silicone rubber (LSR)) is provided to the bearing tube 7220 that is structured and arranged to support and retain the bearing 7021, and an elastomeric bearing seat or sleeve 7260 (e.g., comprising an elastomeric material, e.g., TPE, TPU, TPV, and LSR) is provided to the end wall 7210 that is structured and arranged to support and retain the bearing 7022. In an example, each of the bearing seats 7240, 7260 comprises LSR which may be less susceptible to creep-fatigue and adheres well to plastic substrates.

In the illustrated example, e.g., see FIGS. 28A and 28B, the bearing seat 7240 is supported and retained by the bearing tube 7220 and a base wall 7222 that extends radially inwardly from an end of the bearing tube 7220. The bearing seat 7240 includes a cylindrical or tubular side wall 7242 providing a cylindrical opening to support and retain the bearing 7021. The cylindrical side wall 7242 includes an elongated configuration, and an upper side of the side wall 7242 includes one or more annular bumps or ribs 7245 (e.g., 2, 3, 4, or more bumps or ribs) for retaining the bearing 7021 in an operative position. As illustrated, the bumps or ribs 7245 are configured and arranged to engage along an outer race of the bearing 7021. The inner race of the bearing 7021 is configured and arranged to engage the rotor 7005.

In the illustrated example, the lower side of the side wall 7242 (adjacent the base wall 7222) is devoid of any bumps or ribs, which lower side protrudes past the bearing 7021 and provides a space for enclosing and positioning a spring or biasing element 7300. As illustrated, the spring or biasing element 7300 is arranged between the base wall 7222 and the bearing 7021 to apply a pre-load force to the bearing 7021 and/or maintain alignment of the magnet 7010 with the stator assembly 7015.

In the illustrated example, e.g., see FIGS. 27A and 27B, the bearing seat 7260 is supported and retained by the end wall 7210. The bearing seat 7260 includes a cylindrical or tubular side wall 7262 providing a cylindrical opening to support and retain the bearing 7022. The cylindrical side wall 7262 includes one or more annular bumps or ribs 7265 (e.g., 2, 3, 4, or more bumps or ribs) for retaining the bearing 7022 in an operative position. As illustrated, the bumps or ribs 7265 are configured and arranged to engage along an outer race of the bearing 7022. The inner race of the bearing 7022 is configured and arranged to engage the rotor 7005.

The elastomeric bearing seats 7240, 7260 are arranged between the stator 6040 and respective bearings 7021, 7022, e.g., to isolate vibrations, reduce noise, and provide shock absorption, e.g., in the radial direction.

The elastomeric bearing seats 7240, 7260 may be permanently (e.g., overmolded) or removably (e.g., interference fit assembly) connected to the stator 6040. In an example, the elastomeric bearing seats 7240, 7260 may include one or more retaining structures configured to provide an interference fit or mechanical interlock with the stator 6040 when the elastomeric bearing seats 7240, 7260 are overmolded to the stator 6040.

In some forms, one or more aspects of the elastomeric bearing seats or sleeves 7240, 7260 may be similar to examples described in WO 2021/178527, which is incorporated herein by reference in its entirety.

In the illustrated example, e.g., see FIGS. 25 and 26, the end wall 7210, the bearing tube 7220, and respective elastomeric bearing seats 7240, 7260 are configured and arranged to support and align bearings 7021, 7022 of different sizes relative to one another, which aligns the rotor 7005 with the axis of the blower 6000. As illustrated, the bearing seat 7240 is configured to support bearing 7021 and bearing seat 7260 is configured to support bearing 7022 having a larger size or diameter than bearing 7021. For example, the bearing seat 7240 forms a diameter to support bearing 7021, and bearing seat 7260 forms a larger diameter to support bearing 7022. In alternative examples, bearings with similar sizes relative to one another may be used.

As illustrated, the magnet 7010 is positioned between the bearings 7021, 7022 such that the magnet 7010 is aligned with the stator assembly 7015.

In an example, a spacer 7310 may be provided between the bearing 7021 and the magnet 7010 and a spacer 7320 may be provided between the bearing 7022 and the magnet 7010. The spacers 7310, 7320 along with the spring 7300 are configured to maintain alignment of the magnet 7010 with the stator assembly 7015. In an example, the rotor 7005, the magnet 7010, the bearings 7021, 7022, and the spacers 7310, 7320 may form a rotor assembly 7004 (e.g., see FIG. 22), which rotor assembly 7004 forms a sub-assembly that is pre-assembled prior to assembly to other components of the blower 6000.

A rotor cap or end cap 7400 is provided to the end wall 7210 of the stator 6040. The rotor cap 7400 is configured to engage and provide a stop for the bearing 7022, and hence retains the rotor 7005 to the stator 6040 and within the bearing tube 7220. The rotor cap 7400 and the base wall 7222 each form an opening configured to allow respective end portions of the rotor 7005 to extend therethrough.

In the illustrated example, the stator 6030 comprises end wall 7510, a cylindrical outer wall 7530, and a hub 7535 provided to the end wall 7510. In the illustrated embodiment, the end wall 7510 and the outer wall 7530 may be integrally formed (e.g., injection molded of plastic material) as a one-piece structure, and the hub 7535 (e.g., comprising an elastomeric material) may be formed separately from the end wall 7510 and the outer wall 7530 and then connected to the end wall 7510 (e.g., overmolded, interference fit assembly). As described above, a plurality of stator vanes 7532 (e.g., four stator vanes) are provided to the outer wall 7530 to direct the flow of air from the impeller 6050 along the stator 6030 to the blower outlet 6098. In the illustrated example, as described above, a central portion 7533 of each stator vane 7532 may be hollow, or recessed from the surrounding outer portions of the respective stator vane 7532.

As shown in FIG. 25, the stators 6030, 6040 are configured to engage with one another, and cooperate to support and maintain the stator assembly 7015 in an operative position. For example, the bearing tube 7220 and the outer walls 7230, 7530 form a substantially cylindrical space to receive the stator assembly 7015, and cooperate to enclose and sandwich the stator assembly 7015 in an operative position.

In the illustrated example, e.g., see FIGS. 21-24 and 26, the outer wall 7530 of the stator 6030 includes a plurality of openings 7539 (e.g., four openings) spaced around its perimeter, and the outer wall 7230 of the stator 6040 includes a plurality of openings 7239 (e.g., four openings) spaced around its perimeter. As illustrated, each of the openings 7529, 7239 includes a square shape, however other shapes are possible. The stator assembly 7015 (e.g., the exterior surface of the lamination stack 7018) is exposed to the gas flow along respective stators 6030, 6040 via the openings 7539, 7239 in the stators 6030, 6040, which allows forced-convection cooling of the stator assembly 7015 as gas flows through the blower to the blower outlet 6098 in use.

FIGS. 29A, 29B, 29C-1, 29C-2, 29D, 29E-1, 29E-2, 29F, 29G-1, and 29G-2 show an assembly procedure for the blower 6000 shown in FIGS. 20 to 28B according to an example of the present technology.

For example, as shown in FIG. 29A, the stator 6040 is formed along with its elastomeric bearing seats 7240, 7260, e.g., elastomeric bearing seats 7240, 7260 comprise an overmolded connection to the stator 6040.

Then, as shown in FIG. 29B, the stator assembly 7015 may be pre-formed and inserted onto the exterior surface of the bearing tube 7220 of the stator 6040. In an example, a varnish coating may be applied to the stator assembly 7015, e.g., to insulate and protect the windings.

As shown in FIGS. 29C-1 and 29C-2, following insertion of the stator assembly 7015, the stator 6030 is connected or otherwise assembled to the stator 6040 into an assembled configuration, which supports and maintains the stator assembly 7015 in an operative position. As illustrated, the hub 7535 of the stator 6030 is configured to engage the exterior surface of the bearing tube 7220. In addition, the base rings of the stators 6030, 6040 contact one another and are secured to one another in any suitable manner, e.g., heat staking, welding (e.g., ultrasonic welding), adhesive, fasteners. For example, the stator 6030 may be secured to the stator 6040 via heat staking, e.g., the stator 6030 includes stakes or posts 7550 (e.g., four stakes or posts) along the perimeter of its base ring configured and arranged to extend through respective openings 7250 along the perimeter of the base ring of stator 6040 and subsequently heat staked to align and secure the stator 6030 to the stator 6040.

After the stators 6030, 6040 are in an assembled configuration, the rotor assembly 7004 (i.e., sub-assembly including the rotor 7005, the magnet 7010, the bearings 7021, 7022, and the spacers 7310, 7320) and the spring 7300 are inserted into the stator 6040 and the bearing tube 7220 thereof as shown in FIG. 29D. In an example, the spring 7300 is first inserted or dropped into the bearing tube 7220 through the upper end of the bearing tube 7220, and the spring 7300 is configured to engage the base wall 7222 that extends radially inwardly from the lower end of the bearing tube 7220 which provides a stop or support for the spring 7300 at the lower end. Following insertion of the spring 7300, the side of the rotor assembly 7004 including the smaller bearing 7021 is inserted or dropped into the bearing tube 7220 through the upper end of the bearing tube 7220. As the smaller bearing 7021 reaches the lower end of the bearing tube 7220, the smaller bearing 7021 engages the one or more annular bumps or ribs 7245 of the bearing seat 7240 which supports and retains the bearing 7021 in an operative position, while the larger bearing 7022 engages the one or more annular bumps or ribs 7265 of the bearing seat 7260 which supports and retains the bearing 7022 in an operative position. As illustrated, the spring 7300 is arranged between the base wall 7222 and the bearing 7021 to apply a pre-load force to the bearing 7021 and/or maintain alignment of the magnet 7010 with the stator assembly 7015.

As shown in FIGS. 29E-1 and 29E-2, following insertion of the rotor assembly 7004 and the spring 7300 into the stator 6040, the rotor cap 7400 is connected or otherwise assembled to the stator 6040 into an assembled configuration, which supports and maintains the rotor assembly 7004 in an operative position, e.g., rotor cap 7400 configured to engage the bearing 7022 which provides a stop to retain the rotor assembly 7004 within the bearing tube 7220. As illustrated, in the assembled configuration, end portions of the rotor 7005 are configured to extend through respective openings formed in the rotor cap 7400 and the base wall 7222. In addition, the rotor cap 7400 and the end wall 7210 of the stator 6040 are secured to one another in any suitable manner, e.g., heat staking, welding, adhesive, fasteners. For example, the rotor cap 7400 may be secured to the end wall 7210 via heat staking, e.g., the end wall 7210 includes stakes or posts 7270 (e.g., six stakes or posts) along its perimeter configured and arranged to extend through respective openings 7405 along the perimeter of the rotor cap 7400 and subsequently heat staked to align and secure the end wall 7210 to the rotor cap 7400.

As shown in FIG. 29F, after the rotor cap 7400 is connected to the stator 6040, the impeller 6050 and the shroud 6070 are installed (e.g., interference or press fit) to the respective end portion of the rotor 7005 and cooperate to form a complete double-shrouded impeller for one side of the blower. Similarly, the impeller 6060 and the shroud 6080 are installed (e.g., interference or press fit) to the respective end portion of the rotor 7005 and cooperate to form a complete double-shrouded impeller for the other side of the blower. As described above, the impeller and shroud may be formed separately (e.g., injection molded) from one another (i.e., multi-part construction including 2 separate parts), and then the two parts may be assembled separately to the rotor 7005 to form the complete double-shrouded impeller. Alternatively, the impeller and shroud may be formed separately (e.g., injection molded) from one another, and then lightly fastened to one another (e.g., via an interlock or adhesive) to form a unit before assembly to the rotor 7005. In yet another alternative, the impeller and shroud may be integrally formed in one piece, and then assembled to the rotor 7005.

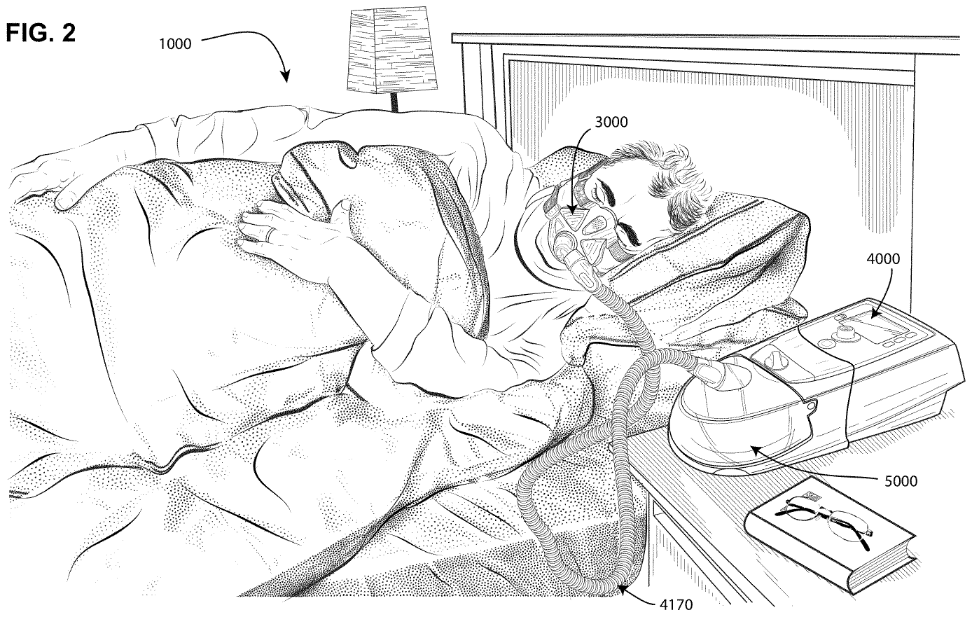

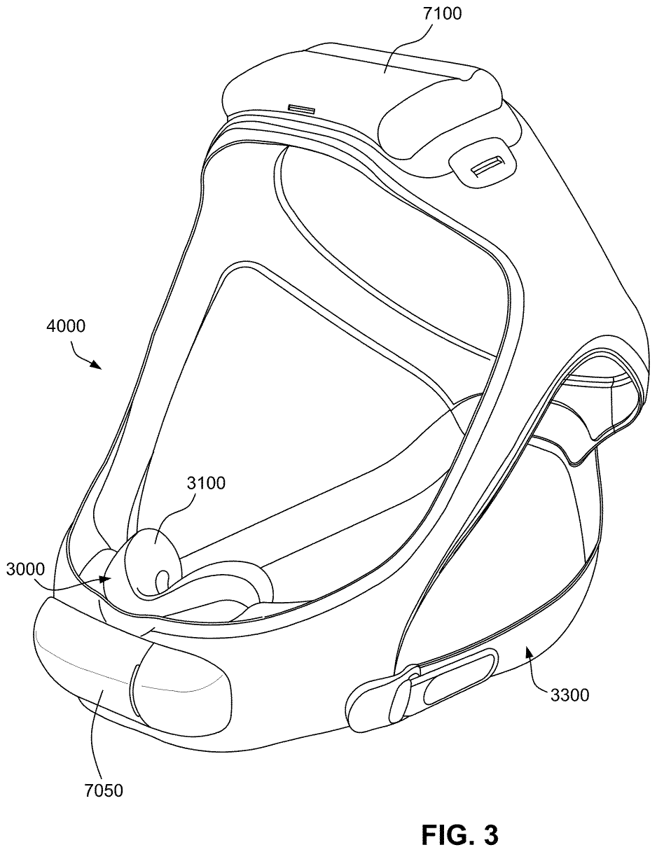

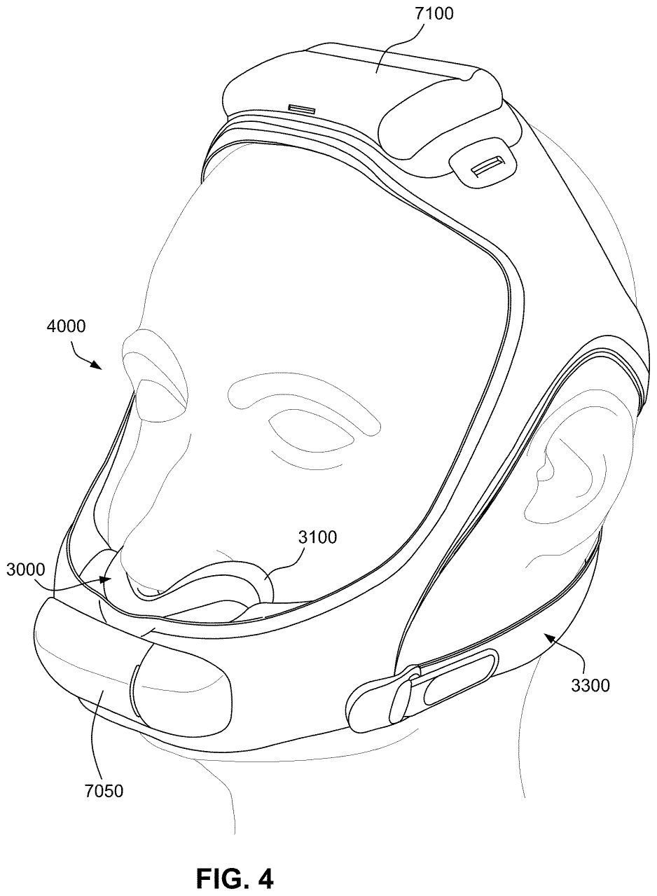

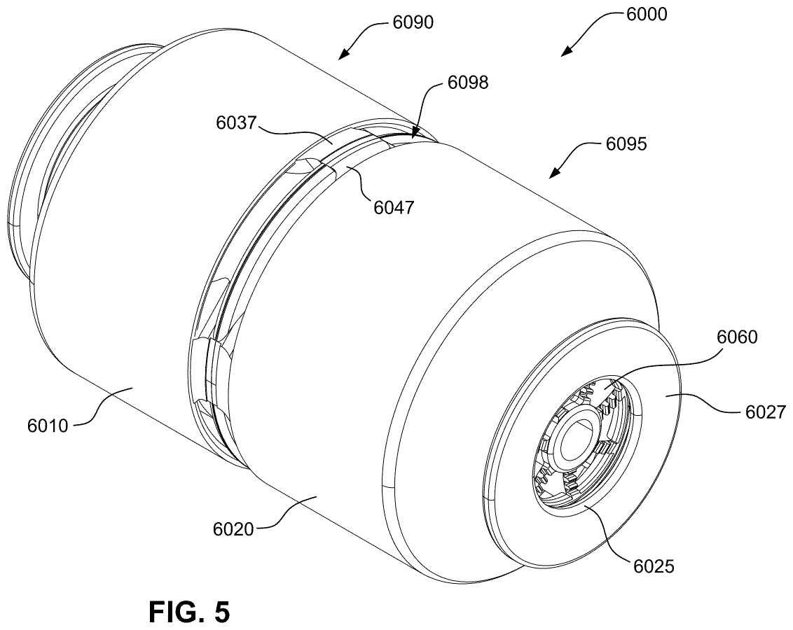

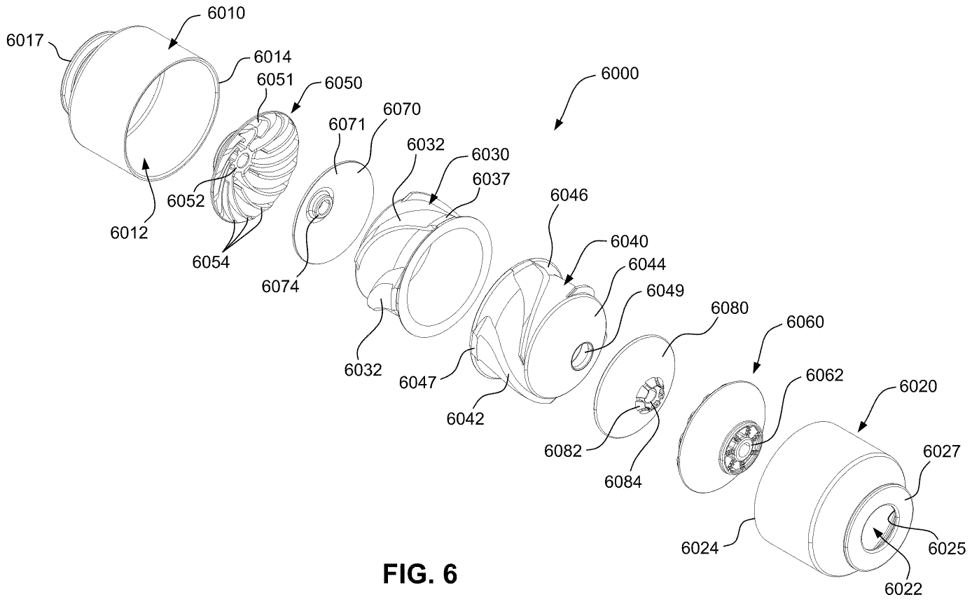

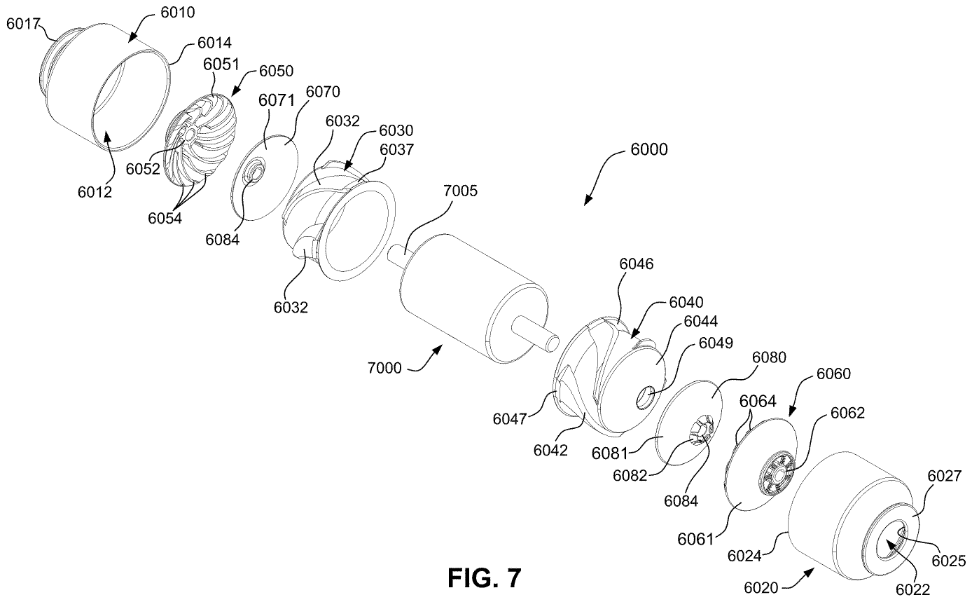

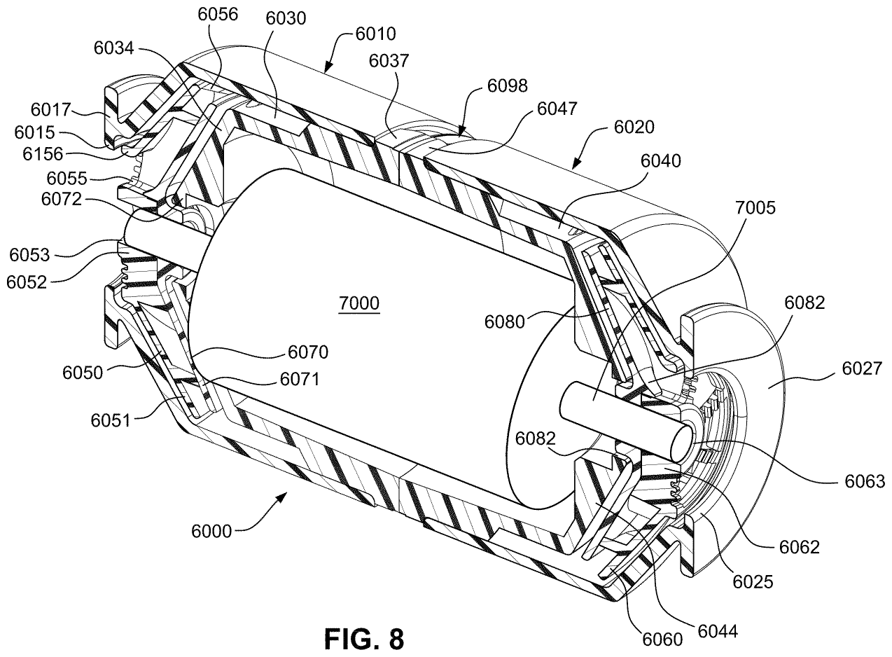

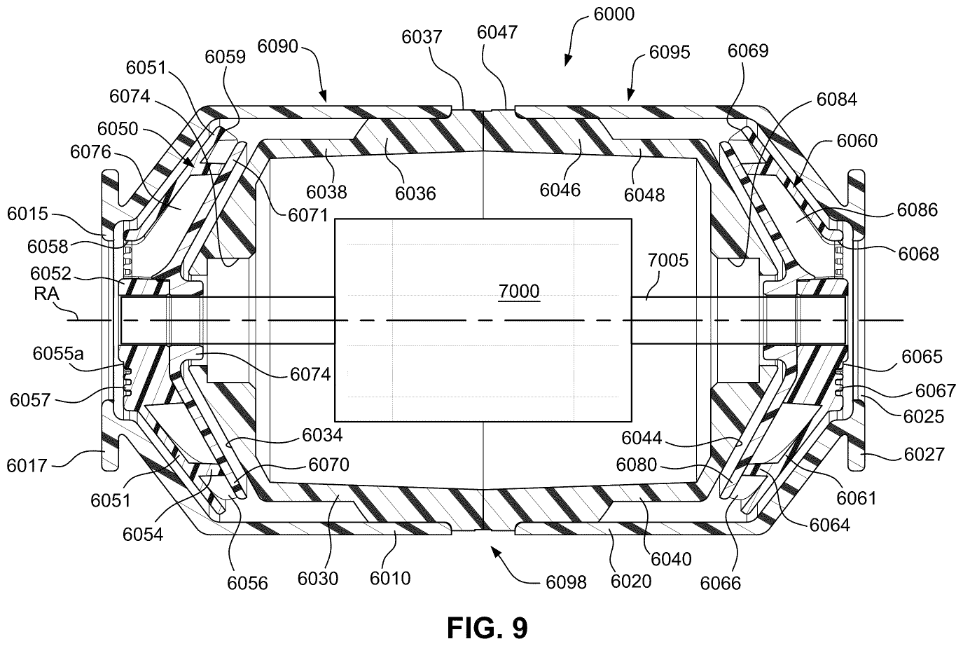

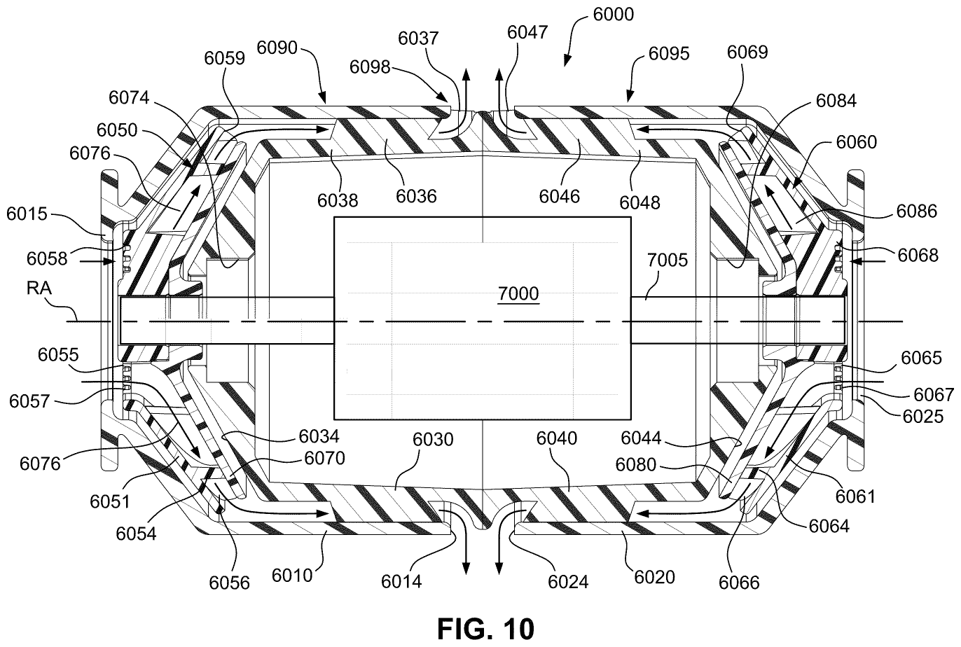

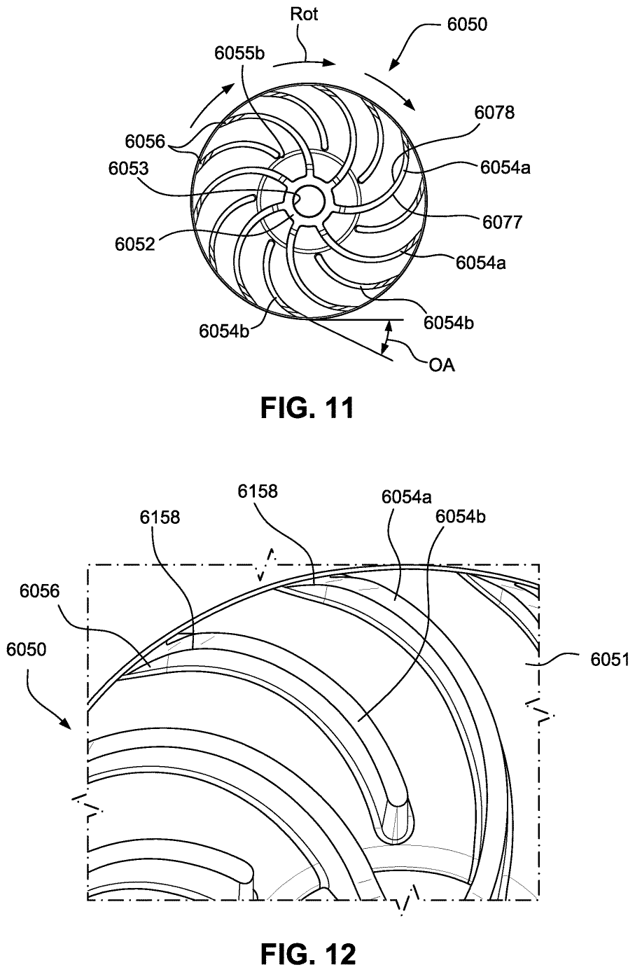

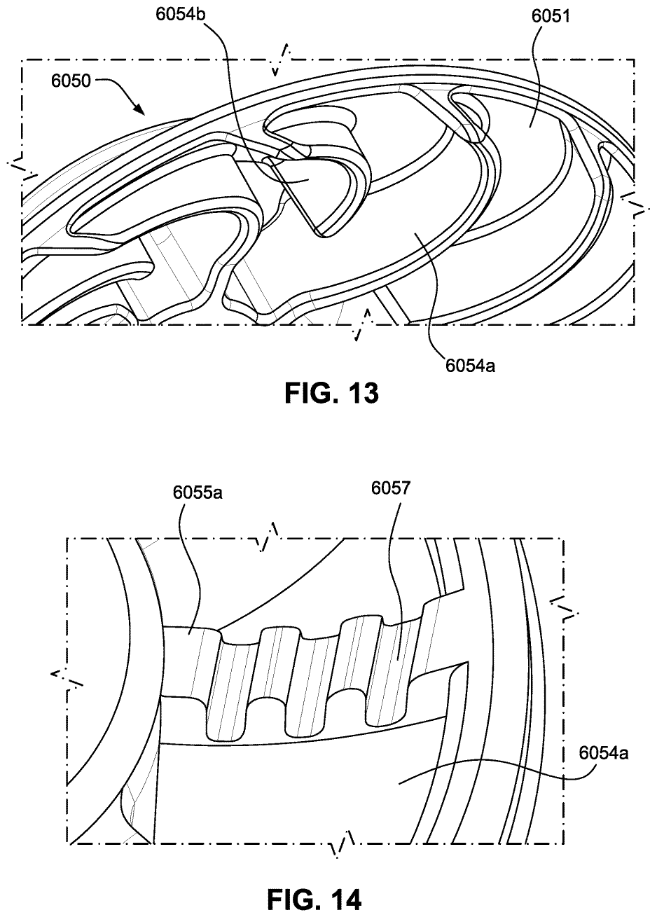

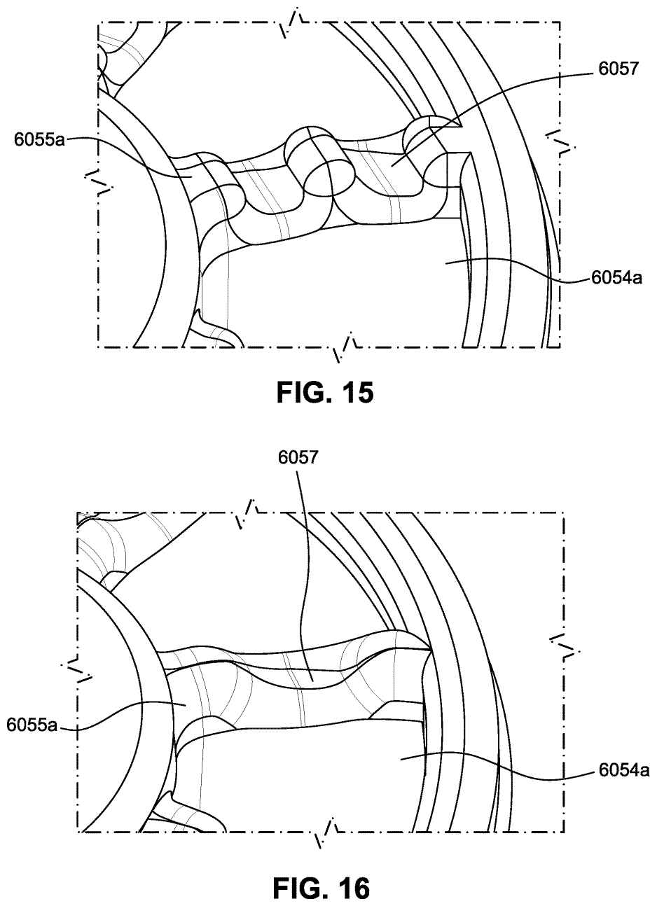

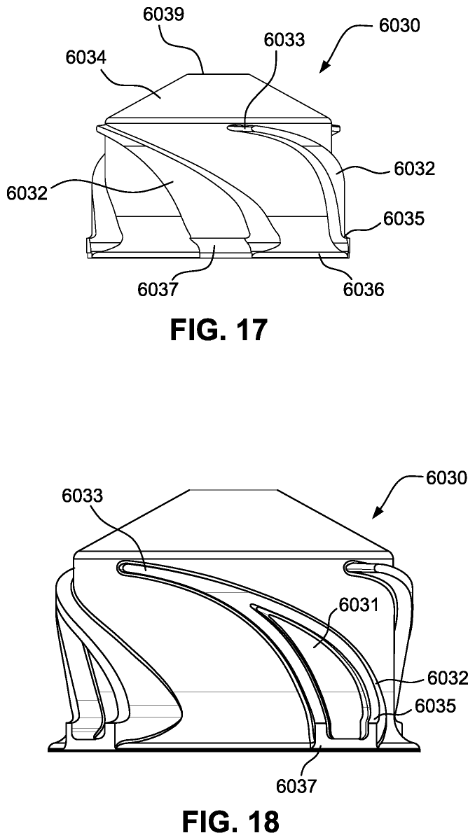

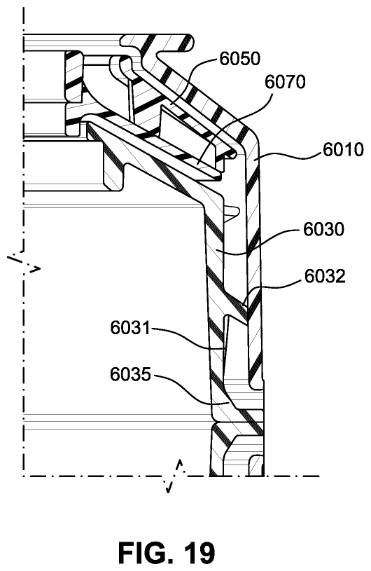

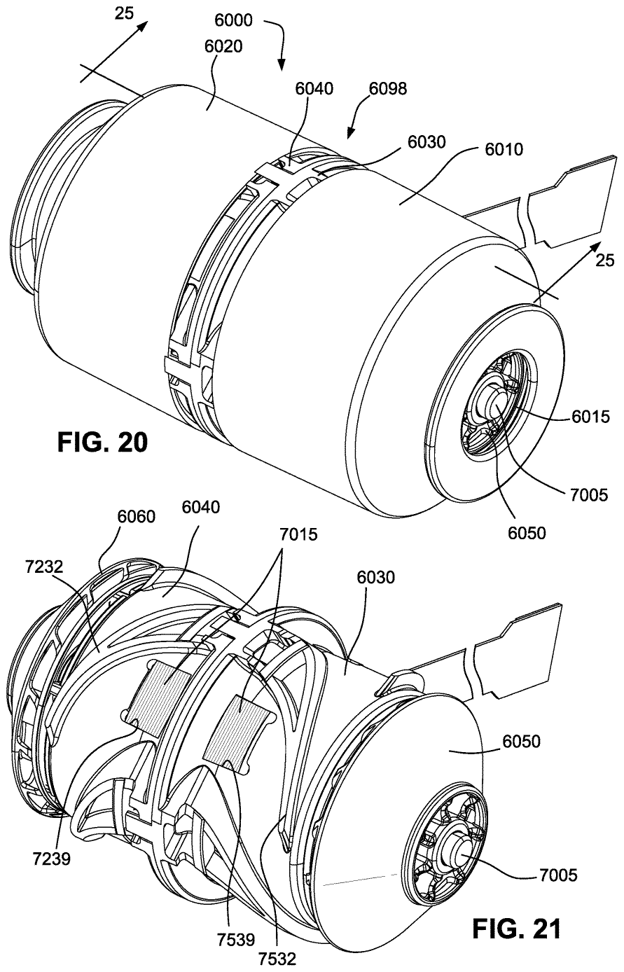

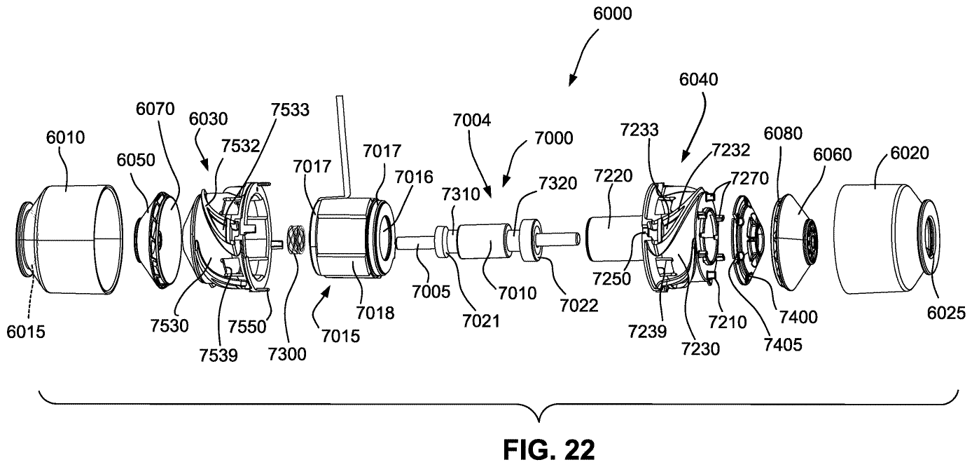

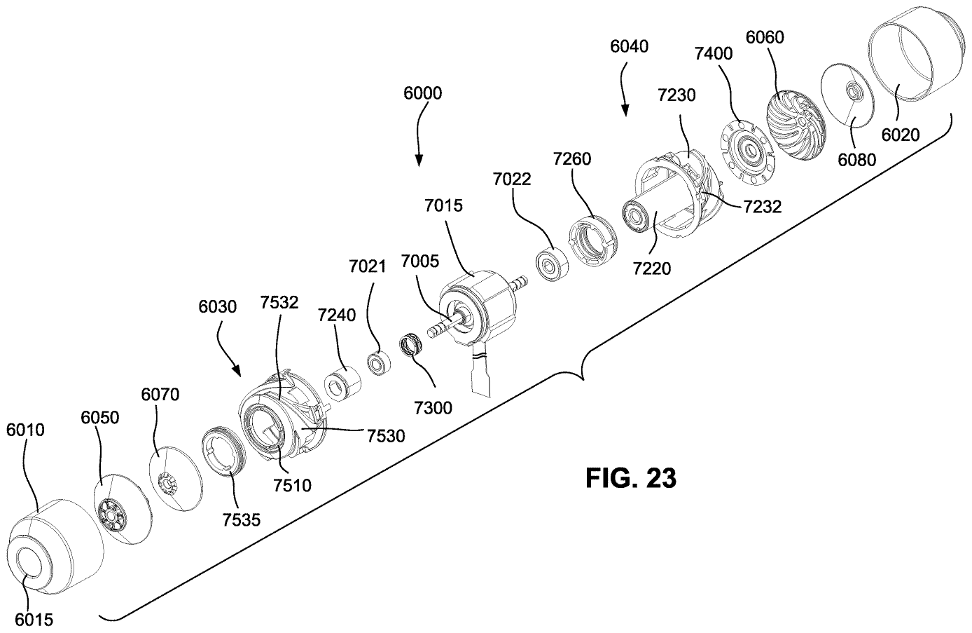

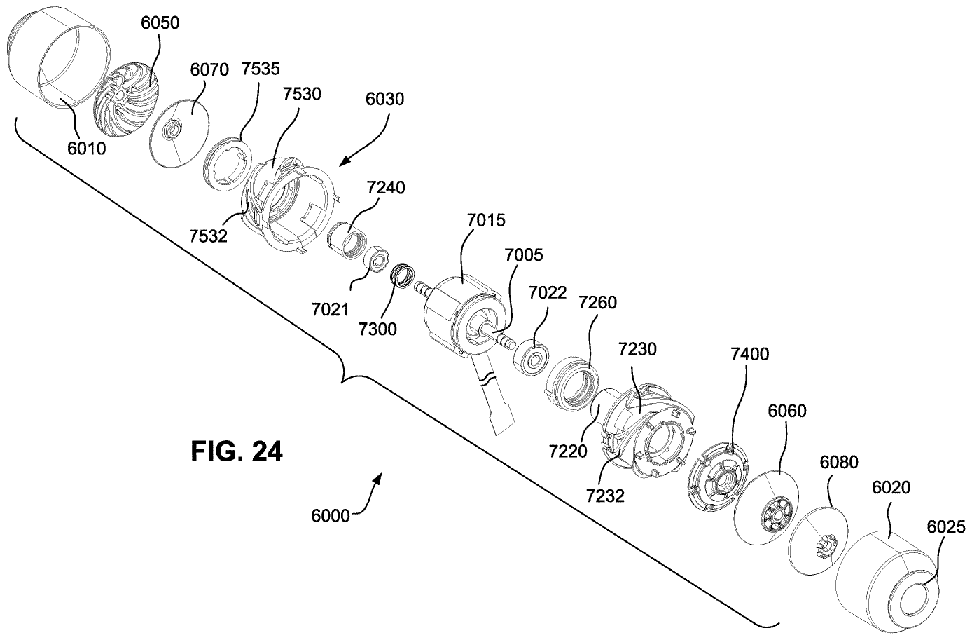

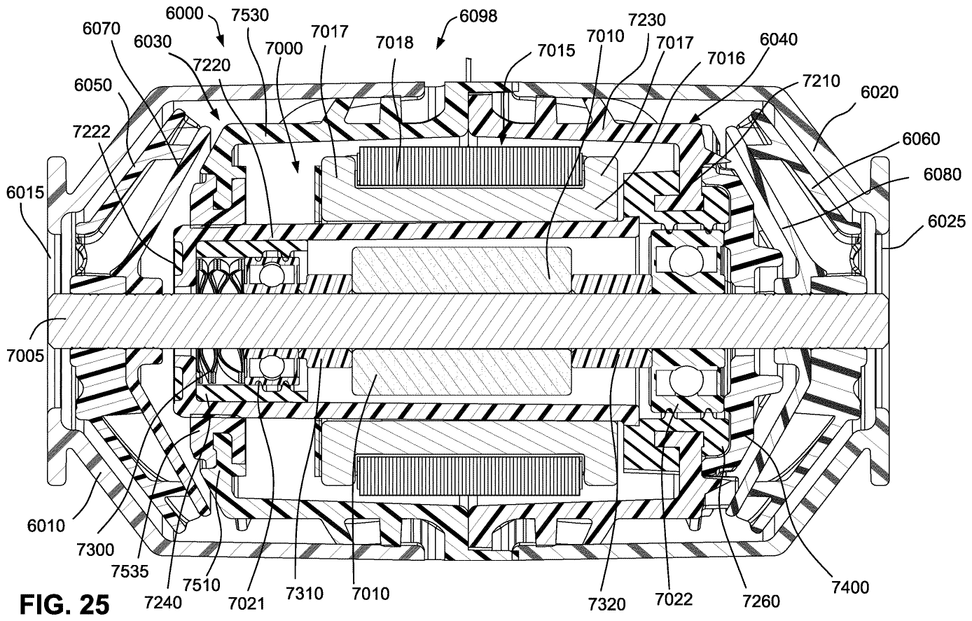

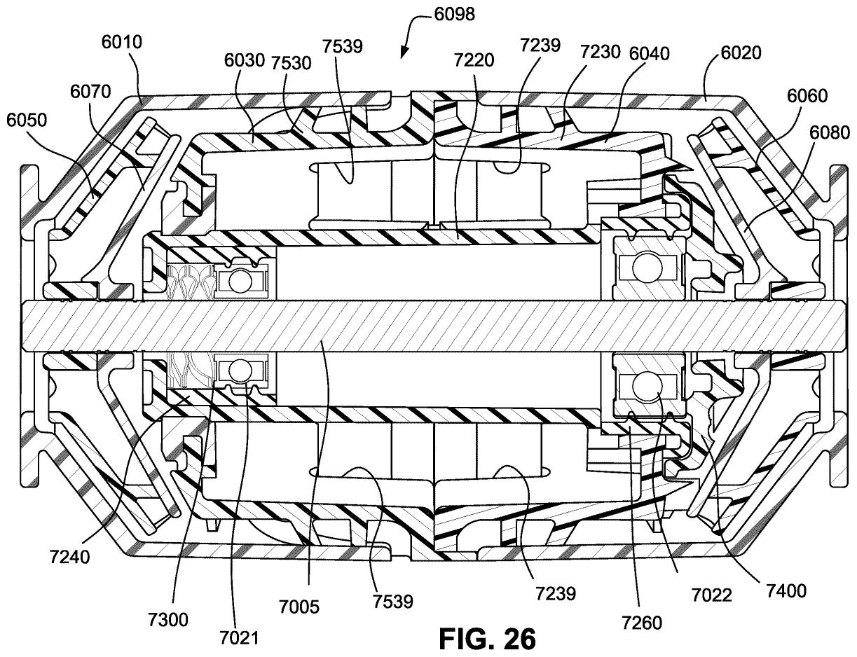

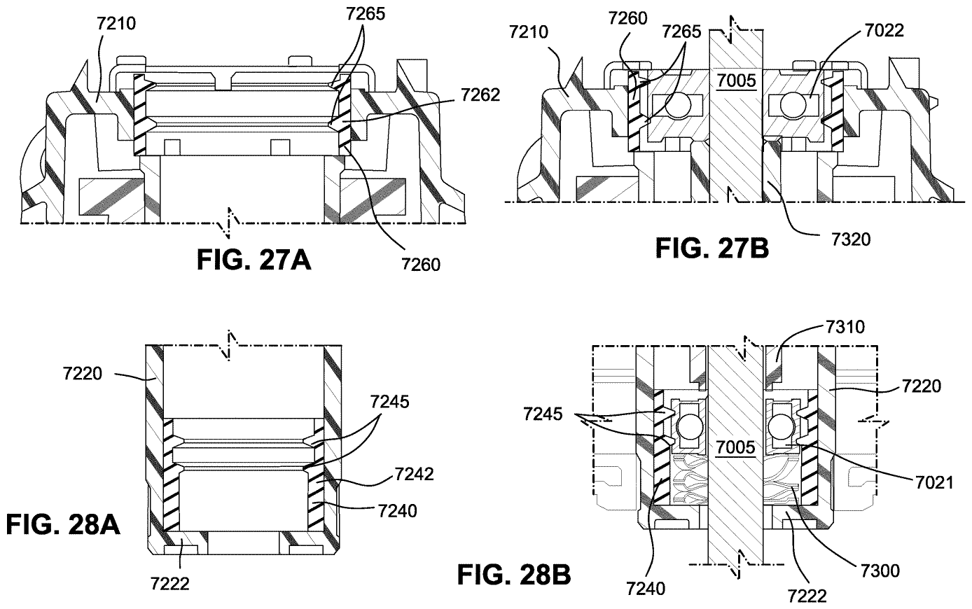

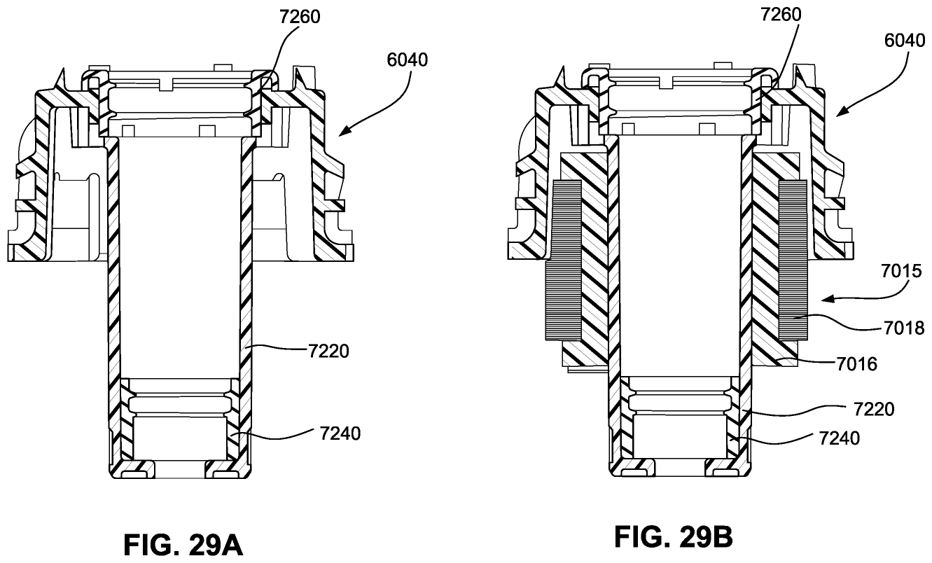

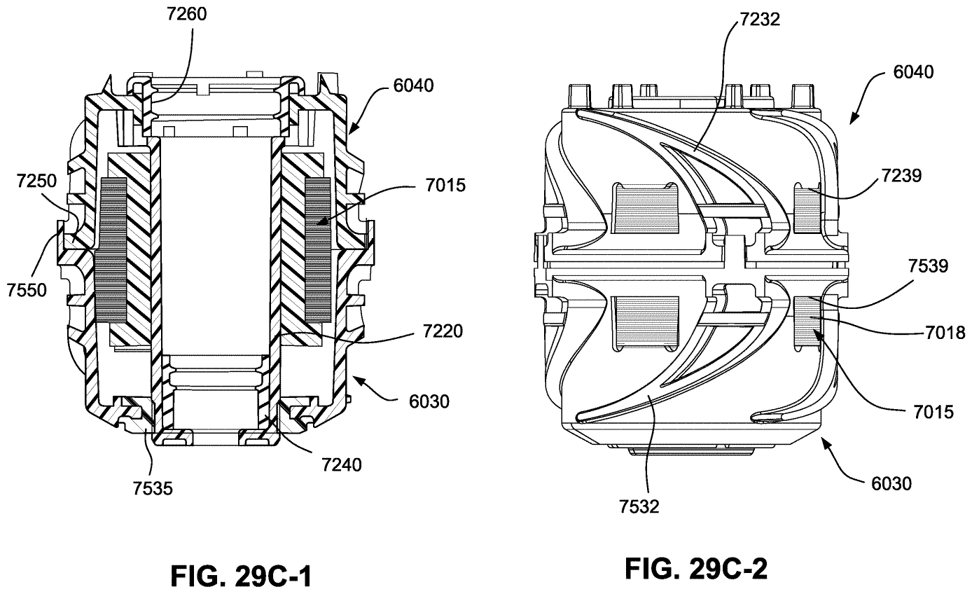

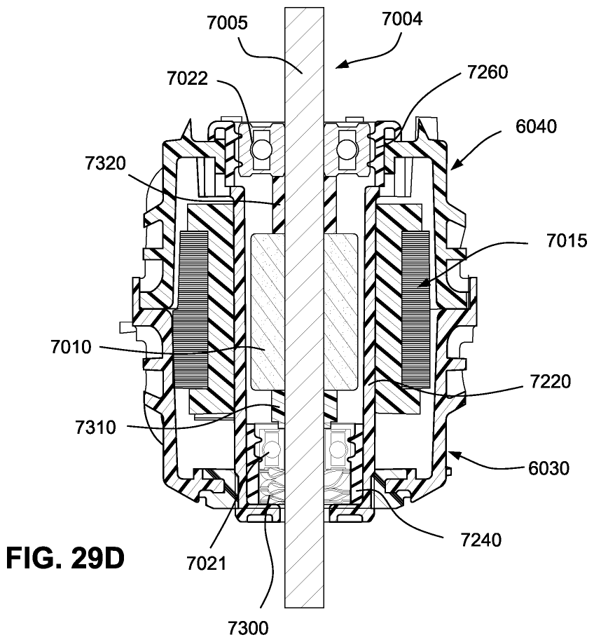

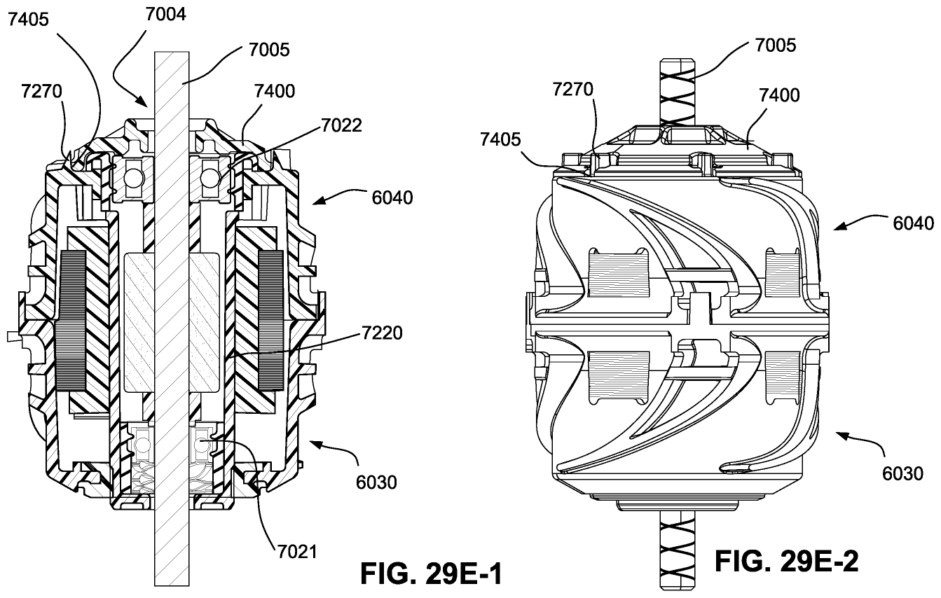

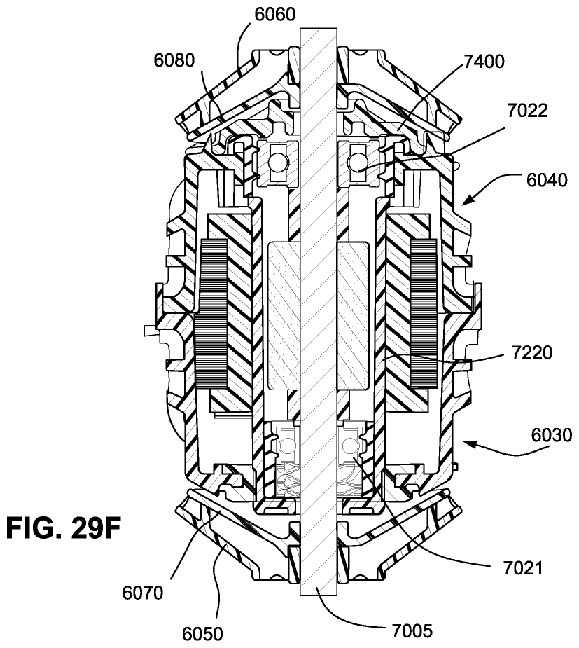

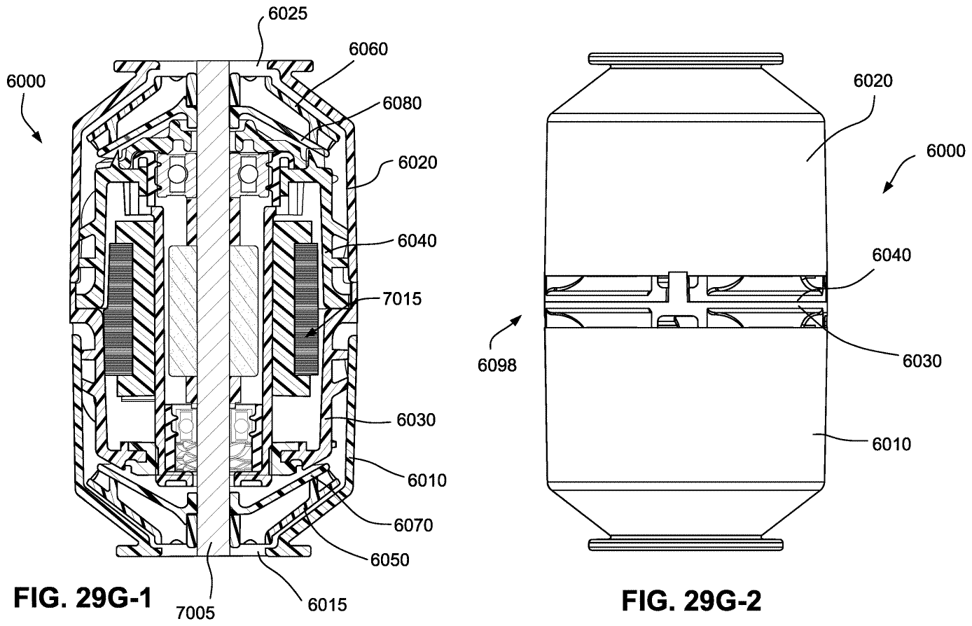

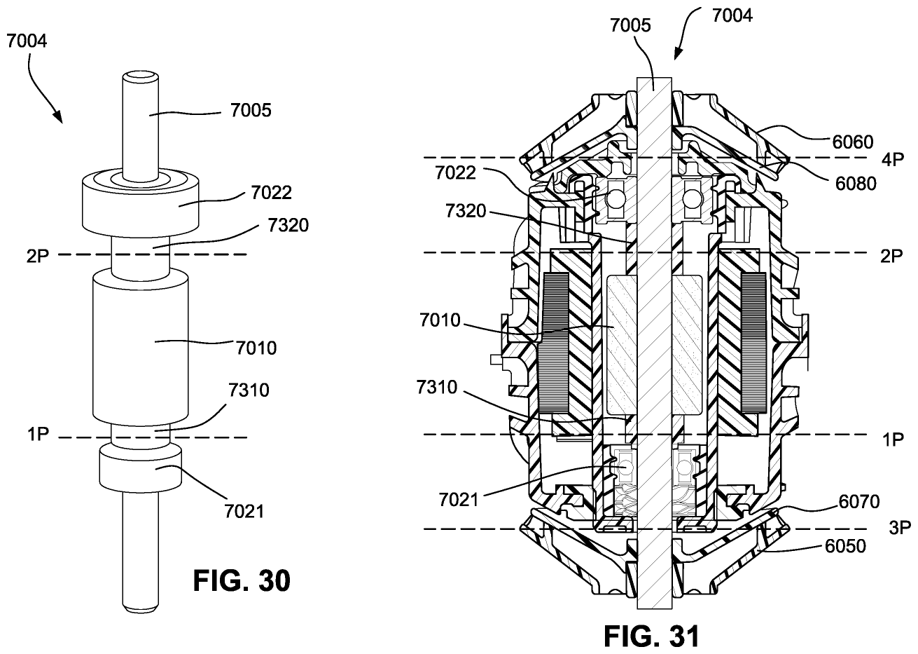

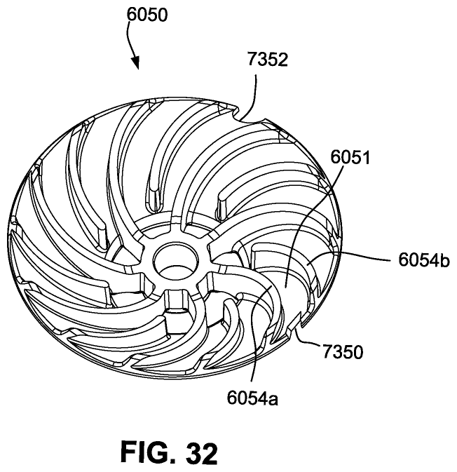

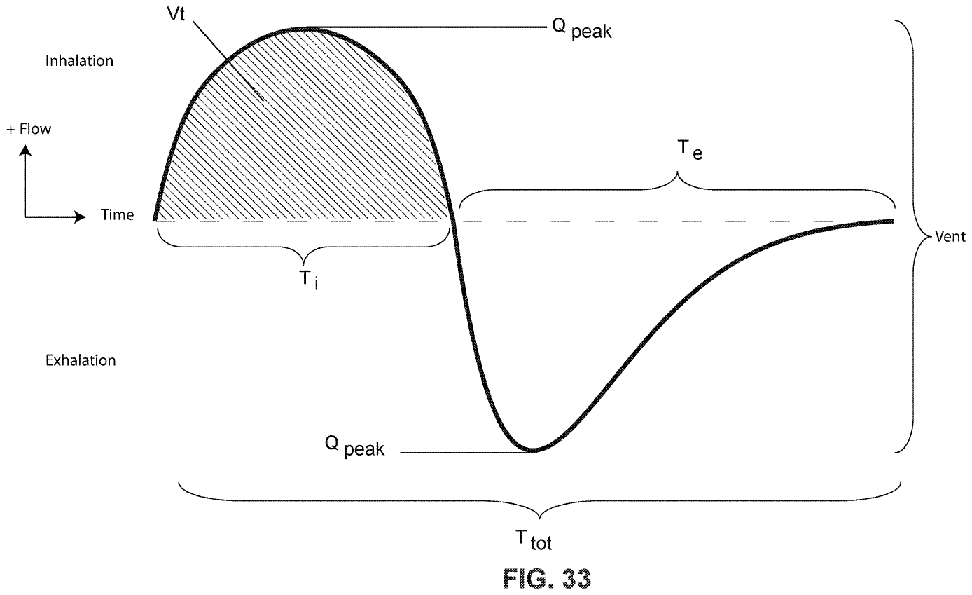

As shown in FIGS. 29G-1 and 29G-2, following assembly of the impellers 6050, 6060 and shrouds 6070, 6080 to the rotor 7005, the housings 6010, 6020 are installed to respective stators 6030, 6040. As illustrated, the housing 6010 forms air inlet 6015 and at least partially houses the stator 6030, the impeller 6050, and the shroud 6070, and the housing 6020 forms air inlet 6025 and at least partially houses the stator 6040, the impeller 6060, and the shroud 6080. As described above, the stators 6030, 6040 may be at least partially positioned outside of the respective housing 6010, 6020, which spaces the housings 6010, 6020 apart from one another to form the blower outlet 6098 (e.g., see FIGS. 20, 25, 26, and 29G-2). In an example, the housings 6010, 6020 may be installed to respective stators 6030, 6040 in any suitable manner, e.g., laser welding.

In an example, a dynamic balancing process may be applied to the rotating masses to minimize noise and vibration during operation. For example, a balancing process may be applied at both a rotor-level and at a blower-level according to an example of the present technology. Because multiple components are attached to the rotor 7005 (e.g., bearings, spacers, impellers, etc.), the center of mass may be offset from a geometric center of the rotor 7005 (i.e., an imbalance), which creates noise and/or vibration. The imbalance may be reduced by reducing mass so that the center of mass will more closely align to the geometric center of the rotor 7005.

Before the rotor assembly 7004 (i.e., sub-assembly including the rotor 7005, the magnet 7010, the bearings 7021, 7022, and the spacers 7310, 7320) is inserted into the stator 6040 and the bearing tube 7220 thereof (as shown in FIG. 29D), a balancing process may be applied to the rotor assembly 7004, i.e., balancing at a rotor-level.

As shown in FIG. 30, balancing at a rotor-level may be accomplished, for example, by running or rotating the rotor 7005 at an operating speed and measuring any imbalance (e.g., wobble), reducing any imbalance by mass removal from one or both of two balancing planes (e.g., a first balancing plane 1P through the spacer 7310 and a second balancing plane 2P through the spacer 7320), running the rotor 7005 again at an operating speed and remeasuring any imbalance, and repeating mass removal accordingly. In example, mass may be removed from one or both balancing planes 1P, 2P by removing mass from one or both spacers 7310, 7320, e.g., drilling one or more radial holes or notches in one or both spacers 7310, 7320 depending on the degree and location of imbalance. In an example, balancing at the rotor-level may be performed multiple times (e.g., 2, 3, 4, or more times) to reduce imbalance.

After the impellers 6050, 6060 and the shrouds 6070, 6080 are installed to the respective end portions of the rotor 7005 (as shown in FIG. 29F), a balancing process may be applied to the blower, i.e., balancing at a blower-level.

As shown in FIG. 31, balancing at a blower-level may be accomplished, for example, by running or rotating the rotor 7005 at an operating speed and measuring any imbalance (e.g., wobble), reducing any imbalance by mass removal from at least one of four balancing planes (e.g., a first balancing plane 1P through the spacer 7310, a second balancing plane 2P through the spacer 7320, a third balancing plane 3P through the outer diameter of the impeller 6050, and a fourth balancing plane 4P through the outer diameter of the impeller 6060), running the rotor 7005 again at an operating speed and remeasuring any imbalance, and repeating mass removal accordingly. In example, mass may be removed from balancing plane 1P by removing mass from spacer 7310, mass may be removed from balancing plane 2P by removing mass from spacer 7320, mass may be removed from balancing plane 3P by removing mass from impeller 6050, and/or mass may be removed from balancing plane 4P by removing mass from impeller 6060, depending on the degree and location of imbalance. In an example, as shown in FIG. 32, mass from the outer diameter impeller 6050 (or likewise for impeller 6060) may be removed by creating a notch or cut-out (e.g., size of notch or cut-out depending on degree of imbalance or wobble) in a portion of the shroud 6051 between primary and secondary blades 6054a, 6054b. For example, a notch 7350 may be created on the outer diameter of the impeller 6050 (or likewise for impeller 6060) by a clipping tool (e.g., similar to a nail clipper) or a notching tool, e.g., by clipping a notch in a portion of the shroud 6051 or chipping off material in a portion of the shroud 6051. In another example, a notch or cut-out 7352 may be created on the outer diameter of the impeller 6050 (or likewise for impeller 6060) by a drilling tool, e.g., drilling or grinding off a portion of the shroud 6051. In an example, balancing at the blower-level may be performed multiple times (e.g., 2, 3, 4, or more times) to reduce imbalance.

In an example, imbalance may be reduced by reducing mass from the spacers and/or the impellers. In an alternative example, imbalance may be reduced by adding mass to the spacers and/or the impellers.

In an example, because the rotor 7005 operates at high rotational speeds necessary for outputting therapeutically effective pressure, an aggressive balancing process is applied at both a rotor-level (e.g., mass removal at least one of two planes) and at a blower-level (e.g., mass removal at least one of four planes) to reduce imbalance so as to minimize noise and vibration during operation.

An air circuit 4170 in accordance with an aspect of the present technology is a conduit or a tube constructed and arranged to allow, in use, a flow of air to travel between two components such as blower 6000 to the patient interface 3000. In an example embodiment such as that depicted in FIGS. 3 and 4, the blower enclosure links directly to the patient interface 3000 chamber such no additional air circuit is needed. In an alternative example, blower 6000 could be mounted in an enclosure located elsewhere on the positioning and stabilising structure 3300, such as for example, adjacent to (or in place of the battery 7100) located near the crown. In such an example, internal passages inside the positioning and stabilising structure 3300 could link the blower and the patient interface 3000, forming the air circuit 4170.

In such an example, one or more heating elements may be situated about the passages forming the air circuit to, for example, maintain or raise the temperature of the air. The heating element may be in a form of a heated wire circuit, and may comprise one or more transducers, such as temperature sensors. In one form, the heated wire circuit may be helically wound around the passages. The heating element may be in communication with a controller such as a central controller such as that described in U.S. Pat. No. 8,733,349, which is incorporated herewithin in its entirety by reference.

### 5.2 Breathing Waveforms

FIG. 33 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.3 Glossary

For the purposes of the present technology disclosure, in certain forms of the present technology, one or more of the following definitions may apply. In other forms of the present technology, alternative definitions may apply.

### 5.3.1 General

Air: In certain forms of the present technology, air may be taken to mean atmospheric air, and in other forms of the present technology air may be taken to mean some other combination of breathable gases, e.g., oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH2O, g-f/cm² and hectopascal. 1 cmH2O is equal to 1 g-f/cm² and is approximately 0.98 hectopascal (1 hectopascal=100 Pa=100 N/m²=1 millibar ~0.001 atm). In this specification, unless otherwise stated, pressure is given in units of cmH2O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.3.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.3.3 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilizing structure designed to hold a device, e.g. a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.4 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.5 Reference Signs List

| | |
|---|---|
| patient | 1000 |
| patient interface | 3000 |
| seal-forming structure | 3100 |
| positioning and stabilising structure | 3300 |
| RPT device | 4000 |
| air circuit | 4170 |
| humidifier | 5000 |
| blower | 6000 |
| left housing | 6010 |
| left cavity | 6012 |
| left cavity opening | 6014 |
| left air inlet | 6015 |
| flat surface | 6017 |
| right housing | 6020 |
| right cavity | 6022 |
| right cavity opening | 6024 |
| right air inlet | 6025 |
| flat surface | 6027 |
| left stator | 6030 |
| stator vanes | 6032 |
| top ring | 6034 |
| base ring | 6036 |
| outlet rib | 6037 |
| outer wall | 6038 |
| hub | 6039 |
| right stator | 6040 |
| stator vane | 6042 |
| top ring | 6044 |
| base ring | 6046 |
| outlet rib | 6047 |
| outer wall | 6048 |
| hub | 6049 |
| left impeller | 6050 |
| outer surface | 6051 |
| left hub | 6052 |
| hub opening | 6053 |
| left blade | 6054 |
| leading edge | 6055 |
| trailing edge | 6056 |
| serrations | 6057 |
| impeller inlet | 6058 |
| impeller outlet | 6059 |
| right impeller | 6060 |
| right hub | 6062 |
| hub opening | 6063 |
| right blade | 6064 |
| leading edge | 6065 |
| trailing edge | 6066 |
| serrations | 6067 |
| impeller inlet | 6068 |
| impeller outlet | 6069 |
| left bottom shroud | 6070 |
| shroud clip | 6072 |
| shroud hub | 6074 |
| flow path | 6076 |
| right bottom shroud | 6080 |
| shroud clip | 6082 |
| shroud hub | 6084 |
| flow path | 6086 |
| first compression stage | 6090 |
| second compression stage | 6095 |
| blower outlet | 6098 |
| motor | 7000 |
| rotor assembly | 7004 |
| motor shaft | 7005 |
| magnet | 7010 |
| stator assembly | 7015 |
| windings | 7016 |
| end turn | 7017 |
| lamination stack | 7018 |
| bearing | 7021 |
| bearing | 7022 |
| enclosure | 7050 |
| battery | 7100 |
| end wall | 7210 |
| bearing tube | 7220 |
| base wall | 7222 |
| outer wall | 7230 |
| stator vanes | 7232 |
| central portion | 7233 |
| opening | 7239 |
| bearing seat | 7240 |
| side wall | 7242 |
| ribs | 7245 |
| openings | 7250 |
| bearing seat | 7260 |
| side wall | 7262 |
| ribs | 7265 |
| stakes | 7270 |
| spring | 7300 |
| spacer | 7310 |
| spacer | 7320 |
| notch | 7350 |
| rotor cap | 7400 |
| openings | 7405 |
| end wall | 7510 |
| outer wall | 7530 |
| stator vanes | 7532 |
| central portion | 7533 |
| hub | 7535 |
| opening | 7539 |
| stakes | 7550 |

1. A blower comprising:
a motor configured to drive a motor shaft, the motor shaft having a first end and a second end extending opposite the first end;
a first housing including a first inlet;
a first impeller connected to the first end of the motor shaft, the first impeller configured to be rotated by the motor shaft to deliver a first flow of air from the first inlet toward an outlet,
the first impeller comprising:
a plurality of impeller blades, each of the impeller blades is curved along at least a portion of its length such that each of the impeller blades curves away from a direction of rotation of the first impeller; and
a top shroud and a bottom shroud that define a flow passage therebetween through the first impeller,
the top shroud including a center opening which provides an impeller inlet to allow air to enter the flow passage of the first impeller;
a second housing including a second inlet; and
a second impeller connected to the second end of the motor shaft, the second impeller configured to be rotated by the motor shaft to deliver a second flow of air from the second inlet toward the outlet,
the second impeller comprising:
a plurality of impeller blades, each of the impeller blades is curved along at least a portion of its length such that each of the impeller blades curves away from a direction of rotation of the second impeller; and
a top shroud and a bottom shroud that define a flow passage therebetween through the second impeller,
the top shroud including a center opening which provides an impeller inlet to allow air to enter the flow passage of the second impeller;
wherein the plurality of impeller blades for each of the first and second impellers includes primary blades and secondary blades, and wherein each of the primary blades are longer than each of the secondary blades,
wherein the first flow generated by the first impeller and the second flow generated by the second impeller are configured to flow parallel and opposite to one another along at least a portion of their flow path toward the outlet, and the first and second flows are combined at the outlet, and
wherein the flow passage of each of the first impeller and the second impeller is configured to produce a mixed flow of air having both an axial and centrifugal component.

2. The blower of claim 1, wherein at least some of the plurality of blades of the first impeller and at least some of the plurality of blades of the second impeller include a concave surface and a convex surface, and wherein the convex surface is a leading surface oriented towards the direction of rotation.

3. The blower of claim 1, wherein each blade of the plurality of blades includes a constant curvature.

4. The blower of claim 1, wherein each of the secondary blades are disposed between a pair of primary blades, and wherein each of the primary blades are disposed between a pair of secondary blades.

5. The blower of claim 1, wherein each of the first impeller and the second impeller includes a hub, and wherein the primary blades extend substantially between the hub and an outer perimeter of each impeller.

6. The blower of claim 5, wherein the secondary blades extend substantially to the outer perimeter of each impeller and are spaced apart from the hub.

7. The blower of claim 1, wherein the primary blades and the secondary blades have substantially the same radius of curvature.

8. The blower of claim 1, wherein the first impeller and the second impeller each include an outer diameter between about 20 mm to about 30 mm.

9. The blower of claim 1, wherein the first impeller and the second impeller are conical or frustoconical in shape.

10. The blower of claim 1, wherein the first impeller and the second impeller are substantially identical.

11. The blower of claim 1, wherein the first inlet and the second inlet are coaxial, and the outlet is generally perpendicular to the first and second inlets.

12. The blower of claim 1, wherein the top shroud and the bottom shroud comprise two separate parts.

13. The blower of claim 12, wherein the plurality of impeller blades comprise a once piece construction with the top shroud.

14. The blower of claim 1, further comprising a first stator arranged downstream of the first impeller and a second stator arranged downstream of the second impeller.

15. The blower of claim 14, wherein each of the first and second stators comprises stator vanes to direct the flow of air from the first and second impellers to the outlet.

16. The blower of claim 15, wherein each of the stator vanes includes a surface configured to redirect the flow of air from a generally axial direction to a generally radial direction at the outlet.

17. The blower of claim 14, wherein the motor includes a magnet mounted on the motor shaft and a stator assembly that surrounds the motor shaft and the magnet thereof, wherein the first and second stators are configured to support and maintain the stator assembly in an operative position, and wherein each of the first and second stators comprises a plurality of openings configured and arranged to expose at least a portion of the stator assembly to the flow of air to allow cooling of the stator assembly.

18. The blower of claim 14, further comprising a pair of bearings to rotatably support the motor shaft, wherein one of the first and second stators comprises a pair of bearing seats configured to support and retain a respective one of the pair of bearings, and wherein each of the pair of bearing seats comprises an elastomeric material.

19. The blower of claim 18, wherein each of the pair of bearing seats comprises one or more bumps or ribs configured to engage an outer race of the respective one of the pair of bearings.

20. The blower of claim 14, further comprising a pair of bearings to rotatably support the motor shaft, wherein the motor includes a magnet mounted on the motor shaft and a stator assembly that surrounds the motor shaft and the magnet thereof, wherein one of the first and second stators comprises a bearing tube, and wherein the stator assembly is provided along an exterior surface of the bearing tube and the pair of bearings are provided along an interior of the bearing tube to support the motor shaft and the magnet within the interior.

21. The blower of claim 1, further comprising a pair of bearings to rotatably support the motor shaft, wherein each of the pair of bearings is in the form of a rolling element bearing comprising ceramic ball bearings.

22. The blower of claim 1, further comprising a pair of bearings to rotatably support the motor shaft, wherein the motor includes a magnet mounted on the motor shaft and a stator assembly that surrounds the motor shaft and the magnet thereof, further comprising a pair of spacers, each of the pair of spacers arranged between the magnet and a respective one of the pair of bearings, and wherein the motor shaft, the magnet, the pair of bearings, and the pair of spacers form a sub-assembly.

23. The blower of claim 22, wherein the sub-assembly comprises a rotor-level balanced construction prior to connection of the first impeller and the second impeller to respective first and second ends of the motor shaft.

24. The blower of claim 23, wherein the rotor-level balanced construction comprises mass removal from at least one of two planes each of which extends through a respective one of the pair of spacers.

25. The blower of claim 22, wherein the sub-assembly, the first impeller connected to the first end of the motor shaft, and the second impeller connected to the second end of the motor shaft comprises a blower-level balanced construction.

26. The blower of claim 25, wherein the blower-level balanced construction comprises mass removal from at least one of four planes each of which extends through a respective one of the pair of spacers, the first impeller, and the second impeller.

27. The blower of claim 26, wherein mass removal from the first impeller and/or the second impeller comprises a notch in a portion of the top shroud between adjacent impeller blades.

28. The blower of claim 1, wherein the first and second impellers are configured such that the first flow and the second flow are substantially similar to one another.

29. The blower of claim 1, wherein the blower only includes the first impeller and the second impeller.

30. A blower comprising:
a motor configured to drive a motor shaft, the motor shaft having a first end and a second end extending opposite the first end;
a first housing including a first inlet;
a first impeller connected to the first end of the motor shaft, the first impeller configured to be rotated by the motor shaft to deliver a first flow of air from the first inlet toward an outlet,
the first impeller comprising:
a plurality of impeller blades; and
a top shroud and a bottom shroud that define a flow passage therebetween through the first impeller,
the top shroud including a center opening which provides an impeller inlet to allow air to enter the flow passage of the first impeller;
a second housing including a second inlet; and
a second impeller connected to the second end of the motor shaft, the second impeller configured to be rotated by the motor shaft to deliver a second flow of air from the second inlet toward the outlet,
the second impeller comprising:
a plurality of impeller blades; and
a top shroud and a bottom shroud that define a flow passage therebetween through the second impeller,
the top shroud including a center opening which provides an impeller inlet to allow air to enter the flow passage of the second impeller;
wherein the plurality of impeller blades for each of the first and second impellers includes primary blades and secondary blades, and wherein each of the primary blades are longer than each of the secondary blades,
wherein the first flow generated by the first impeller and the second flow generated by the second impeller are configured to flow parallel and opposite to one another along at least a portion of their flow path toward the outlet, and the first and second flows are combined at the outlet, and
wherein the flow passage of each of the first impeller and the second impeller is configured to produce a mixed flow of air having both an axial and centrifugal component.

31. The blower of claim 30, wherein each of the impeller blades of the first and second impellers is curved along at least a portion of its length such that each of the impeller blades curves away from a direction of rotation of the first and impellers.

32. The blower of claim 30, wherein each of the first impeller and the second impeller includes a hub, and wherein the primary blades extend substantially between the hub and an outer perimeter of each impeller, and wherein the secondary blades extend substantially to the outer perimeter of each impeller and are spaced apart from the hub.

33. The blower of claim 30, wherein the blower only includes the first impeller and the second impeller.
